(19) United States
(12) Patent Application Publication
(10) Pub. No.: US 2016/0159653 A1
(43) Pub. Date: Jun. 9, 2016

(54) HIGH ANISOTROPY NANOPARTICLES

(71) Applicant: Virginia Commonwealth University, RIchmond, VA (US)

(72) Inventors: Everett Carpenter, Mechanicsville, VA (US); Ahmed A. El-Gendy, Richmond, VA (US); Shiv Khanna, Richmond, VA (US)

(73) Assignee: Virginia Commonwealth University, Richmond, VA (US)

(21) Appl. No.: 14/227,283

(22) Filed: Mar. 27, 2014

Related U.S. Application Data
(63) Continuation-in-part of application No. PCT/US2013/020214, filed on Jan. 4, 2013.
(60) Provisional application No. 61/806,238, filed on Mar. 28, 2013.
(60) Provisional application No. 61/703,460, filed on Sep. 20, 2012.
(60) Provisional application No. 61/662,053, filed on Jun. 20, 2012.
(60) Provisional application No. 61/582,963, filed on Jan. 4, 2012.

(51) Int. Cl. C01B 31/30; A61K 41/00; A61K 33/24

(52) U.S. Cl. CPC C01B 31/303; A61K 33/24; A61K 41/0052; C01P 2004/64; C01P 2006/42

(57) ABSTRACT

New nanoparticles are provided which may have blocking temperatures exceeding 570 K even for particles as small as 8 nm in size. First principles theoretical investigations show that the new behavior is rooted in the giant magnetocrystalline anisotropies due to controlled mixing between carbon p-states and cobalt d-states. Furthermore, assemblies of the new nanoparticles may provide rare earth free permanent magnets having magnetic properties which rival those of rare earth permanent magnets.

### CROSS-REFERENCE TO RELATED APPLICATIONS

This application is a continuation-in-part (CIP) application of Patent Cooperation Treaty (PCT) Application No. PCT/US2013/020214 filed Jan. 4, 2013, and which claims the priority of U.S. Provisional Patent Application No. 61/582,963 filed Jan. 4, 2012, U.S. Provisional Patent Application No. 61/662,053 filed Jun. 20, 2012, and U.S. Provisional Patent Application No. 61/703,460 filed Sep. 20, 2012. The present application further claims the benefit of U.S. Provisional Patent Application No. 61/806,238, filed Mar. 28, 2014. All of the aforementioned applications are herein incorporated by reference.

### STATEMENT REGARDING FEDERALLY SPONSORED RESEARCH OR DEVELOPMENT

This invention was made with government support under Grant No. 0472-1574 awarded by the Advanced Research Projects Agency (ARPA), MRI Grants No. CHE0820945 and No. CHE0922582 awarded by National Science Foundation, and Grant No. 0472-1572 awarded by the Advanced Research Projects Agency-Energy (ARPA-e) of the U.S. Department of Energy. The government has certain rights in the invention.

### BACKGROUND

1. Field of the Invention

The invention generally relates to magnetic nanoparticles and permanent magnets made therefrom. In particular, the magnetic nanoparticles include but are not limited to particle sizes of 10 nm and smaller which break or redefine existing superparamagnetic limits.

2. Background of the Invention

Permanent magnets (PMs), especially those containing rare earth metals, are an indispensible component of many applications in electric, electronics, communications, and automobile industries. The emergence of green technology markets such as plug-in hybrid/electric vehicles (e.g. PHEVs and EVs), direct drive wind turbine power systems, and energy storage systems (e.g. flywheels) has created an increased demand for PMs, since they produce high torque with a much smaller motor.

The majority of the cost for producing electric motors is directly related to the magnetic materials used therein, particularly the rare earth metals that are currently used to produce PMs. Unfortunately, recent market trends have made the production and procurement of rare earth permanent magnets more challenging and less cost efficient. The lack of a secure supply chain for rare earth metals makes them very expensive. If electric motors are to achieve prominent integration into green technologies and be affordable for the average consumer, it will be essential to reduce the cost of the materials. Unfortunately, there are currently no existing alternatives to rare earth metals for producing energetically equivalent PMs. There is thus a pressing need in the art to identify new materials to replace rare earth metals in the manufacture of PMs, as well as efficient, cost effective methods of manufacturing PMs using such materials.

The production of non-rare earth cobalt magnetic nanoparticles using a wet chemical polyol process has been described, e.g. see United States patent application publication 2012/0168670 (Harris), the entire contents of which is hereby incorporated by reference. However, this publication describes only a mixture or admixture of magnetic Co₂C and Co₃C phase cobalt carbides nanoparticles and a method for the scalable manufacturing of those particles.

U.S. Pat. Nos. 5,783,263; 5,549,973; and 5,456,986 (Majetich et al., the entire contents of each of which is hereby incorporated by reference) describe metal carbide nanoparticles. However, the particles are coated and their manufacture is tied exclusively to a particular process that involves preparing graphite rods which are packed with a magnetic metal oxide.

Magnetic nanoparticles are a key to dense memory storage, targeted drug delivery, and a variety of other industrial and medical applications including components in nano-electronic circuits. For a given magnetic material, a reduction in the magnetic domain size smaller than the typical domain size for a bulk object results in a nanomagnet where the atomic moments are exchange coupled and the particle behaves like a giant magnet with a moment Nμ, where N is the number of atoms and μ is the moment per atom. In the case of ultrafine particles, however, the magnetic anisotropy energy responsible for holding the magnetic moment along certain directions becomes comparable to the thermal energy. Thermal fluctuations produce random flipping of the magnetic moment with time and the magnetization of nanoparticles exhibit thermal instability. A key to enhancing thermal stability of magnetic nanoparticles is to enhance the anisotropy.

One approach proposed by Skumryev et al. [V. Skumryev, S. Stoyanov, Y. Zhang, G. Hadjipanayis, D. Givord, and J. Nogués, Nature 423, 850 (2003).] is to generate core shell species having a central pure metal core surrounded by an oxide material. These authors synthesized Co/CoO nanomagnets where the exchange bias between the central ferromagnet and the surrounding antiferromagnetic oxide enhanced the anisotropy. Such particles have blocking temperatures of around 290 K and thus still below room temperature.

An approach suggested by El-Gendry et al. [A. A. El-Gendy, V. O. Khavrus, S. Hampel, A. Leonhardt, B. Buechner, and R. Klingeler. J. Phys. Chem. C 114, 10745 (2010).] suggested generation of core/shell nanoalloys which exhibited an enhanced magnetocrystalline anisotropy in the range of 0.3 to 2.6×10⁵ J/m³ for 8 nm NiRu@C nanoalloy. However, the blocking temperature (T_{B}) of the nanoalloys was in the range from 50 to 200 K which is below room temperature, showing a short magnetic range order.

An outstanding problem in nano-magnetism is to stabilize the magnetic order in nanoparticles at room temperatures and above. For ordinary ferromagnetic materials, a reduction in size leads to a decrease in the magnetic anisotropy, resulting in superparamagnetic relaxations at nanometer sizes, in particular sizes of 10 nm or less. The transition limits the use of nanoparticles for memory storage and other applications and one of the fundamental questions is if the anisotropy could be enhanced in reduced sizes. Novel approaches are needed to have intrinsic increase in anisotropy that can lead to much higher blocking temperatures.

### SUMMARY

Provided are novel single phase magnetic alloy non-rare earth nanoparticles, magnets made therefrom, and methods of making the same in quantities sufficient to be practical for use in manufacturing. The magnetic nanoparticles do not contain any rare-earth metals, and, in some embodiments, they are pure single phase materials. The nanoparticles are advantageously made using continuous flow synthesis systems and methods which produce significantly more nanoparticulate material than do prior art systems and methods. In exemplary embodiments, the continuous flow systems use a polyol synthetic process and/or synthetic processes that employ supercritical liquids. The magnetic metallic materials produced as described herein include, or are used to make, both soft and hard magnets.

Provided herein are single phase magnetic alloy nanoparticles. In some embodiments, the single phase magnetic alloy nanoparticles are comprised of a material selected from the group consisting of: Co₂C, Co₃C, Fe₃C, Fe₅C₂, and Fe₇C₃. In some embodiments, the single phase magnetic alloy nanoparticles do not contain rare earth metals.

Also provided are methods of synthesizing single phase magnetic alloy nanoparticles, the methods comprising steps such as i) introducing one or more fluid solutions comprising a salt of a magnetic metal into a continuous flow microfluidic reactor; ii) subjecting the one or more solutions to reaction conditions which allow the salt of a magnetic metal to form single phase magnetic alloy nanoparticles; and iii) recovering the single phase magnetic alloy nanoparticles by subjecting them to a magnetic force. In some embodiments, one fluid solution is introduced into the continuous flow microfluidic reactor and the reaction conditions include maintaining the fluid solution at a pressure and temperature sufficient to convert the fluid solution to a supercritical fluid (SCF). This embodiment may further comprise a step of releasing the pressure from the SCF in order to cause flash evaporation of the SCF, which leaves behind the nanoparticles. The step of releasing may be carried out prior to a step of collecting the nanoparticles. The method may also include a step of collecting and/or purifying the single phase magnetic alloy nanoparticles after the flash evaporation of SCF, e.g. using a magnetic separating device. In other embodiments, the salt of a magnetic metal is contained in one of two fluid solutions that are introduced into the continuous flow microfluidic reactor, and the method includes a step of mixing the two fluid solutions. The step of mixing is carried out at a temperature and pH and for a period of time sufficient to permit said salt of a magnetic metal to react with other components of the solutions and to form the single phase magnetic alloy non-rare earth nanoparticles.

In yet other embodiments, systems for synthesizing single phase magnetic alloy nanoparticles are provided. The systems comprise, for example: i) a continuous flow reactor; ii) a controller to control conditions within the continuous flow reactor; and iii) a magnetic separation device configured to subject the single phase magnetic alloy nanoparticles to a magnetic field.

Using synthetic chemical methods, new nanoparticles (e.g. nanoparticles of a new cobalt carbide phase) are provided which have blocking temperatures which exceed room temperature (up to values greater than 570 K) even for particles 10 nm or less, less than 10 nm, and at least as small as 8 nm in size. Room temperature is generally regarded as being between 293 K and 296 K. First principles investigations show that the new behavior is rooted in the giant magnetocrystalline anisotropies due to spin orbit coupling, particularly controlled mixing between carbon p-states and cobalt d-states. In exemplary embodiments, nanoparticles are provided with an effective magnetocrystalline anisotropy constant in the boundary inclusive range of 6.5×10⁵ J/m³ to 9×10⁵ J/m³. “Boundary inclusive range” indicates that the specified range includes the values at both the start and the end of the range. Nanoparticles according to exemplary embodiments may be single phase. The current nanoparticles may be used for a new generation of thermally stable data storage devices and when assembled, form strong permanent magnets for a variety of devices such as a device for converting between mechanical energy and electrical energy (i.e. converting mechanical energy to electrical energy or vice versa).

### BRIEF DESCRIPTION OF THE DRAWINGS

FIG. 1. XRD analysis for synthesized Co₃C.

FIG. 2a-2c. Magnetic properties of the synthesized Co₃C. (a) The magnetic hysteresis loops at different temperatures. (b) The coercivity dependence on temperature revealing information regarding T_{B} and H_{C0}. (c) TEM image for the Co₃C nanoparticles.

FIG. 3. Magnetic anisotropy energy ΔE(θ,φ) at two angles φ=0° and φ=90°.

FIG. 4. Band structures along the Γ and X direction for bulk Co₃C (left) and Co₃E (E=empty sphere) (right). The size of the dots is proportional to the contribution from the Co d-states.

FIG. 5. The fluctuation time dependence on temperature; inset plot is showing the two minima of the Eₐₙᵢₛ and the maximum value at θ=90°.

FIGS. 6a-6c. Magnetic properties of the synthesized Co₃C. (a) The temperature dependence of dθ/dt. (b) Curie temperature (T_{C}) dependence on number of atoms for different shapes. (c) The remnant magnetization (Mᵣ) dependence on temperature revealing information regarding the magnetic efficiency loss.

FIG. 7. The temperature dependence on the rotation of the magnetic moment around the easy axis which appears in a circle its radius is the average angular radius (rotation distance) of the Co₃C particles.

FIGS. 8a and 8b. ETXRD scans of Co₂C (a) and Co₃C (b) with CoₓC and Co phases identified. (α=HCP—Co, β=FCC—Co) Co₂C is the only phase present up to 250° C. At 250° C., the Co₂C (111) peak shifts to a lower angle, while the (020) peak of Co2C shifts to the (100) peak of α-cobalt. The (111) peak of Co₂C is present till 350° C. At 375° C. the α-cobalt phase transitions to the β-cobalt phase. Co₃C was the major crystal phase up to 300° C. Also at 275° C. a low intensity for the α-cobalt (101) peak is noticed. Above 300° C. the Co₃C phase transitions to α-cobalt. At this transition, the (210) peak of Co₃C shifts to the (002) peak of α-cobalt and the Co₃C (211) peak evolves to the (101) peak of α-cobalt.

FIGS. 9a and 9b. XPS C 1 s scans of as prepared (a) Co₃C and (b) Co₂C nanoparticles. Inset in each scan is a space filled model of each carbide phase, modeled from collected XRD scans with cobalt shown in black and carbon in gray.

FIGS. 10a-10h. Bright Field TEM images of (a) Co₃C particles, (b-d) Co₃C particle surface showing the presence of a gylcolate layer and fine crystallites, both indicated by arrows, (e-f) HRTEM of Co₃C particles showing glycolate layer and inset FFT corresponding to Co₃C <010> zone axis, (g) Co₂C and (h) Co₃C crystal structures (where gray dots represent cobalt atoms and black dots represent carbon atoms) showing the hard and easy magnetization axis.

FIGS. 11a-11e. Contour plots showing relationship of Co₂C composition and Grain Size on (a) Hc in kOe, (b) Ms in emu/g, and (c) BHmax in MGOe. (d) Isothermal Remanent Magnetization (IRM) plots (solid lines) and DC Demagnetization plots (DCD) (dotted lines) of Co₃C, Co₃C rich, and Co₂C rich nanocomposites. (e) Henkel plots derived from the IRM and DCD values using the equation: δM=MDCD-(1-2MIRM). Positive δM values represent exchange coupling to be the dominant magnetic interactions, while a negative δM signify magnetostatic interactions.

FIGS. 12a-12d. Depiction of XMCD sum rules. (a), Helicity-dependent spectra (μ+ & μ−); (b), XMCD spectrum (μ+−μ−) and XMCD integral; (c), average XAS (μ++μ−) and its integral. The values p,q, and r are the integrals needed in the sum rules. (d), expression for the orbital (morb) and spin (mspin) moments.

FIG. 13. XRD scans for Ni (top), and Co (bottom) particles collected after 2.5 hr. reaction time. Reitveld refinement fitted profiles for the Ni Co are superimposed. Miller indices for the FCC-phases and HCP—Co phase are indicated.

FIGS. 14a-14d. TEM images of (a,b) nickel particles, and (c,d) cobalt particles. Inset shows FFT calculated lattice spacing.

FIG. 15. Thermogravimetric curve for synthesized Co particles.

FIGS. 16a and 16b. (a) Room temperature magnetization vs. applied field curves for synthesized cobalt (black) and nickel (gray) particles. (b) Zero field cooled (dotted) and field cooled (solid) magnetization vs. temperature for cobalt (black) and nickel (gray) particles collected at 250 Oe.

FIG. 17. Schematic representation of a continuous flow system with a magnetic separator.

FIG. 18. Motor/generator with permanent magnets comprising magnetic nanoparticles.

FIG. 19. Magnetic storage media comprising magnetic nanoparticles.

### DETAILED DESCRIPTION

Magnetic alloy non-rare earth nanoparticulate material (nanoparticles) are provided, the nanoparticles being producable by a continuous flow process which results in a pure, single phase material. The nanoparticles comprise non-rare earth metals and/or alloys of non-rare earth metals. In other words, the nanoparticles generally do not contain rare earth metals, rare earth metals are absent from the nanoparticles, and the percentage or content of rare earth metals in the nanoparticles is essentially zero, i.e. they are rare-earth free. Such nanoparticles may be referred to herein as “single-phase magnetic alloy nanoparticles, “magnetic alloy nanoparticulate materials”, “magnetic non-rare earth nanoparticles”, “magnetic nanoparticles of the invention”, “metal nanoparticles”, “nanoparticles”, etc. Magnets made with the non-rare earth magnetic nanoparticles (which may be either hard or soft magnets) may be referred to as “non-rare earth magnets”, “non-rare earth permanent (‘hard’) magnets”, “non-rare earth soft magnets”, etc., or by other equivalent terms or phrases.

As used herein, “alloy” is a material in which two or more elements are combined in a single crystal/crystalline structure. The crystal structure may in some cases be the same as that of the constituents, or may be different from that of the constituents. In exemplary embodiments, the alloys are metal alloys.

As used herein, “coercivity” takes on a materials science context. Specifically, coercivity (coercive field, coercive force) of a ferromagnetic material is the intensity of an applied magnetic field required to reduce the magnetization of that material to zero after the magnetization of the sample has been driven to saturation. Thus, coercivity measures the resistance of a ferromagnetic material to becoming demagnetized.

As used herein, “nanoparticle” is an ultrafine particle sized between 1 and 1000 nanometers. “Sized” generally refers to the smallest dimension of the particle, e.g. diameter if the particle is substantially a rod, spherical, and/or contains circular arcs; or length, width, etc. if the particle is angular (e.g. cubic).

As used herein, “permanent magnet” or “hard magnet” is an object made from a material that is magnetized (magnetic, ferromagnetic material) and creates its own persistent magnetic field. Permanent magnets are made from “hard” ferromagnetic materials that are subjected to special processing in a powerful magnetic field during manufacture, to align the individual atomic magnetic dipoles of the internal microcrystalline structure, making them very hard to demagnetize. When all or substantially all magnetic dipoles in a magnet are aligned, the magnet is said to be “saturated”. To demagnetize a saturated magnet, a certain magnetic field must be applied, and this threshold depends on the coercivity of the respective material. “Hard” materials have high coercivity e.g. typically greater than 1000 Am⁻¹.

As used herein, “rare earth metals” are the fifteen lanthanides (lanthenum, cerium, praseodynium, neodymium, promethium, samarium, europium, gadolinium, terbium, dysprosium, holmium, erbium, thulium, ytterbium, lutetium) plus scandium and yttrium.

As used herein, “single phase” or “pure phase” describes any material which consists of at least 95% of a single crystallographic phase as determined by X-ray diffraction (XRD).

As used herein, “soft magnets” hold their magnetic abilities only temporarily and are easily magnetized by exposing them to electrical current and demagnetized by removing the electrical current. This behavior is referred to as being “paramagnetic”, and soft magnets may interchangeably be referred to as “paramagnetic magnets”. These magnets may be regulated by the flow of current, and control of their magnetization and demagnetization is vital to ensure the reliability of the devices that rely on them. Soft magnets are found in many devices (MP3 players, computers, transformers, relays, inductors etc. and other devices in which current alternates frequently. Soft magnets typically have intrinsic coercivity less than 1000 Am⁻¹. They may be formed from e.g. amorphous nanocrystalline alloys of iron, nickel and/or cobalt with one or more of the following elements: boron, carbon, phosphorous and silicon; or from soft ferrites which are ferrimagnetic with a cubic crystal structure and the general composition MOFe₂O₃, where M is a transition metal such as nickel, manganese or zinc; from nickel-iron alloys (permalloys) with a wide range of compositions, e.g. from 30 to 80 wt % Ni.

As used herein, “supercritical fluid” is a substance that is at a temperature and pressure above its critical point. The “critical point” (“vapor-liquid critical point” or “critical state”) of a substance occurs under conditions such as specific values of temperature, pressure, or composition at which no phase boundaries exist, for example, where distinct liquid and gas or vapor phases do not exist. Under such conditions, a substance can have properties of both gases and liquids, e.g. the ability to effuse through solids like a gas and dissolve materials like a liquid. At or close to the critical point of a substance, small changes in pressure or temperature can result in large changes in density, allowing many properties of a supercritical fluid to be “fine-tuned”.

As used herein, “superparamagnetic limit” refers to a threshold which defines a transition at which a material, substance, composition, or particle starts or stops exhibiting a magnetic order/magnetic behavior. For a substance which exhibits ferromagnetism in bulk form (e.g. Co₃C colbalt carbide), the superparamagnetic limit may be given as the smallest particle size possible without a switch from ferromagnetic behavior (in which the magnetic dipole substantially maintains its orientation in the absence of a strong external field) to paramagnetic behavior (in which the magnetic dipole moment randomly flips and reverses orientation in the absence of an external field and thus prohibits preservation of information stored according to the dipole moment orientation). This minimum particle size limitation is a common meaning given to “superparamagnetic limit” in reference to magnetic data storage devices (e.g. hard drives). Below such a particle size limit, particles are no longer usable as permanent magnets and therefore are no long usable in magnetic data storage devices or the like. Alternatively, “superparamagnetic limit” may refer to a temperature threshold below which a particle of some material and/or size exhibits ferromagnetism (i.e. behaves as a permanent magnet) and above which the particle exhibits paramagnetism and thus fails to behave or be usable as a permanent magnet. This temperature threshold for a superparamagnetic material may be referred to as its critical temperature.

For materials which are not superparamagnetic, “Curie temperature (T_{C})” is the temperature threshold above which paramagnetic behavior is observed and below which ferromagnetic behavior is observed. In the case of superparamagnetic materials, however, “Curie temperature” refers to a temperature threshold between paramagnetic behavior and superparamagnetic behavior. For superparamagnetic materials and particles, the Curie temperature (T_{C}) is greater than the so-called “blocking temperature (T_{B})”. Below the blocking temperature, the material or particle exhibits ferromagnetic behavior. Between the blocking temperature and the Curie temperature, the material or particle exhibits paramagnetic behavior. Above the Curie temperature, the material or particle exhibits superparamagnetic behavior. It should be appreciated that a “superparamagnetic” material/particle/nanoparticle does not in all circumstances exhibit superparamagnetic behavior. Rather, the adjective “superparamagnetic” indicates that the material does exhibit superparamagnetic behavior under certain conditions, whereas materials which are not characterizable as “superparamagnetic” never under any condition exhibit superparamagnetic behavior. Generally, superparamagnetic behavior is only exhibited for very small particles (e.g. nanoparticles).

As used herein, the “critical temperature” of a composition, material, or particle (e.g. nanoparticle) is the temperature at which the ferromagnetic ordering is lost. That is to say, up to the critical temperature ferromagnetic behavior is observed. Above the critical temperature, ferromagnetic behavior is not observed. For some embodiments, the critical temperature may be identical to the Curie temperature. For some other embodiments, the critical temperature may be identical to the blocking temperature.

As used herein, “magnetic anisotropy”, “magnetic anisotropy energy (MAE)”, or simply “anisotropy energy” describes the dependence of the internal energy of a composition or particle on the direction of the spontaneous magnetization, creating easy and hard directions of magnetization. The total magnetization of a system will prefer to lie along the easy axis. The energetic difference between the easy and hard axis results from two microscopic interactions: the spin-orbit interaction and the long-range dipolar coupling of magnetic moments. The anisotropy energy arises from the spin-orbit interaction and the possibly partial quenching of the angular momentum. The spin orbit coupling is responsible for the intrinsic (magnetocrystalline) anisotropy while the shape anisotropy is a dipolar contribution. Generally, effective magnetocrystalline anisotropy constants (K_{eff}) for magnetic materials are usually in the range 10²-10⁷ J/m³. This corresponds to an energy per atom in the range 10⁻⁸-10⁻³ eV. As used herein, “MAE”, “Eₐₙᵢₛ”, and “ΔE(θ, φ)” are equivalent expressions for representing magnetic anisotropy. For crystalline compositions, as in the case for most exemplary embodiments discussed herein, “magnetic anisotropy energy” may be equivalently referred to as “magnetocrystalline anisotropy energy”, both terms being interchangeable with the acronym “MAE”.

As used herein, K_{eff} is the effective magnetocrystalline anisotropy constant. When K_{eff} is multiplied to the volume of the particles it becomes the anistropy energy (Eₐₙᵢₛ), i.e. Eₐₙᵢₛ=K_{eff}×V. Experimental determinations of K_{eff} include the range (6.5-8.5)×10⁵ J/m³. The calculated K_{eff} from theory is in the range of (7.8-9)×10⁵ J/m³. Anisotropy energy is calculated theoretically from the contribution of spin-orbit coupling to the total energy by constraining the magnetic moment along various directions characterized by the spherical angles θ and φ, i.e. Eₐₙᵢₛ is function of θ and φ and Eₐₙᵢₛ=ΔE(θ, φ)=MAE.

According to exemplary embodiments of the present invention, alternatives for enhancing magnetic anisotropy as compared to approaches such as the use of core-shell configurations are provided. Through chemical synthetic methods, new phases of metal carbide may be created which exhibit novel properties for magnetic nanoparticles of very small sizes, in particular 10 nm or less down to at least 8 nm. In exemplary embodiments, metal carbides are provided which comprise transition metal layers separated by intervening layers of carbon atoms. The transition metal layers may have greater separation than in a pure bulk form of the same stoichiometric composition (i.e. composition having the same elements and ratios of atoms of each constituent element). As one example, nanoparticles are provided which substantially consist of Co₃C, there being three cobalt atoms for every one carbon atom. The atomic arrangement within the crystalline structure may differ from existing cobalt carbides and provides new properties which are not apparent or obvious from, for example, Co₃C bulk form objects. Significantly, nanoparticles of 10 nm or less according to teachings herein exhibit an atomic structure and novel properties which are not present in or obvious from various nanoparticles of larger size and/or other atomic/stoichiometric compositions.

According to exemplary embodiments, separate layers of cobalt and carbon in the metal carbide phase result in large anisotropies which are further compounded by partial mixing between carbon and cobalt states (i.e. electron orbitals). Resulting materials comprising the metal carbide phase have unusually large magnetocrystalline anisotropies.

In some exemplary embodiments, the metal carbide is a transition metal carbide, in particular cobalt carbide (Co₃C). More specifically, biocompatible pure phase cobalt carbide (Co₃C) nanomagnets are synthesized with a particle size as small as 8 nm and with thermal and time stable long range ferromagnetic order up to 571 K (the superparamagnetic limit). This is a new superparamagnetic limit for particles which are 10 nm or smaller in size, and in particular particles as small as 8 nm, allowing for a new generation of thermally stable magnetic data storage devices and other applications involving nanomagnets. First principles investigations highlight the role of structure and composition in providing this new material behavior. Assemblies of the new cobalt carbide nanoparticles behave as permanent magnets with magnetic characteristics that rival those of rare earth permanent magnets.

Although rare earth metals may optionally be included in compositions according to some embodiments, magnetic nanoparticles as taught herein may be entirely or substantially free of rare earth metals. That is to say, magnetic nanoparticles (e.g. Co₃C nanoparticles) are provided which contain no rare earth metals.

The magnetic behavior of the new nanoparticles is best explained within a model of uniaxial anisotropy. The anisotropy energy of the particle is proportional to sin²θ, where θ is the angle between the magnetization and the easy axis. At absolute zero, the magnetization lies at one of two energy minima (θ equals 0° or 180°). When the temperature is raised above absolute zero, the magnetization direction can fluctuate depending on the thermal energy k_{B}T and the energy barrier K_{eff}V (K_{eff} is the effective crystalline anisotropy and V is the particle volume), that exists at θ=±90. Thus, given the ratio of the energy barrier to k_{B}T, and knowing the resonant frequency, one can compute the average time between random reversals that are strongly dependent on the particle size and temperature. As an example, a factor of 2 change in the particle diameter can change the reversal time from 100 years to 100 nanoseconds.

### Example 1

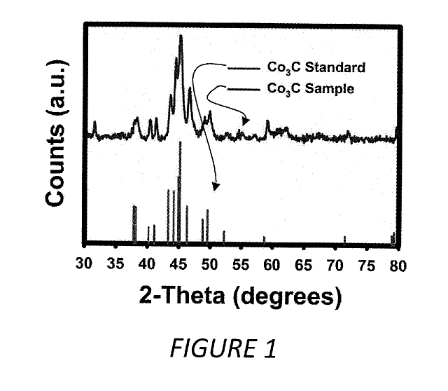

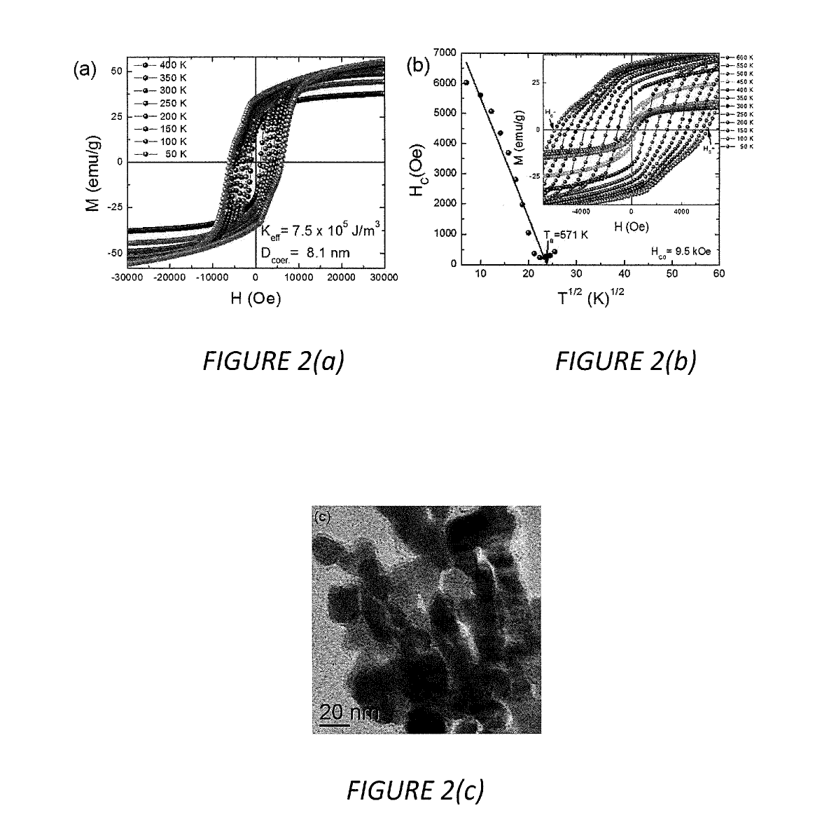

In this study, Co₃C particles were synthesized using a polyol method described in greater detail below and were characterized using x-ray diffractometer to yield 100% of orthorombic-Co₃C nanomagnets. FIG. 1 shows an x-ray diffraction (XRD) analysis for the synthesized Co₃C. The XRD exhibits distinct peaks showing single phase Co₃C nanoparticles. The observed peak broadening reveals a smaller grain size of the particles which can be calculated using the well-known Sheffer equation to be around 11±3 nm. The magnetization dependence on the external magnetic field was measured for the prepared sample at different temperatures ranging from 50 to 400 K (FIG. 2(a)). The observed magnetization shows ferromagnetic behavior for the Co₃C nanomagnet, and there is no knee observed behind the remanence magnetization (Mᵣ) proving the formation of the pure phase carbide in agreement with the result from XRD. The Co₃C shows high coercivity (H_{C}) which increases with decreasing temperature as shown in FIG. 2(b).

Temperature dependent coercivity up to 650K can be used to determine the blocking temperature by using established relations published by Stoner et al. (E. C. Stoner and E. P. Wolfarth, Philos. Trans. R. Soc. London, Ser. A 240, 599 (1948), the complete contents of which are herein incorporated by reference). To this end, the observed coercivity was plotted as a function of T^{1/2} as given in FIG. 2(b). The data reveal blocking temperature T_{B} at H_{C}=0 to be 571 K and the coercivity at zero K (H_{C0}) to be 9.5 kOe. From those results, the effective magnetocrystaline anisotropy (K_{eff}) and the particle size can be determined from Neel Brown equation (e.g. as given by Stoner et al.) to be 7.5±1.0×10⁵ J/m³ and 8.1±0.5 nm, respectively. Nanoparticles according to exemplary embodiments each substantially consist of a single magnetic domain. Thus, a measure of the size of the magnetic domain is also a measure of the particle size. The magnetic domain size can be estimated from the magnetization studies by evaluating the initial slopes of the M(H) curves. Note that the major contribution to the initial slope arises from the largest magnetic domains (i.e. the largest particles). Their larger magnetization vectors are more easily oriented by the magnetic field, and thus an upper limit to the magnetic domain size can be estimated. Further, within a single domain, the anisotropy is dominated by exchange interactions. The observed hysteresis curves show a decrease in the H_{C} till 600 K and an increase thereafter while the saturation magnetization (M_{S}) was decreasing even after 600 K. Such a behavior indicates the presence of long range order and reveals a Curie temperature T_{C} of around 650 K, further indicating no change in the structure as a result of the high temperature measurement at 650 K.

Transmission electron microscope (TEM) measurements were performed to further ascertain the accuracy of the size. The resulting images reveal a narrow distribution of rod nanoparticles with diameter around 10±3 nm in good agreement with the magnetic domain size determined from the magnetic study (FIG. 2(c)). A blocking temperature of 571 K and the effective magnetocrystalline anisotropy of 7.5±1.0×10⁵ J/m³ are both startling findings. This is particularly surprising since bulk cobalt is a soft magnetic material with a magnetic anisotropy of 4.1×10⁵ J/m³. Surprisingly, the anisotropy per Co atom in the new cobalt carbide material is much greater than the anisotropy per Co atom in bulk cobalt since the carbide has a fewer number of Co atoms per unit volume as compared to bulk/pure cobalt. Further, carbon is known to quench the magnetic moment. The observed values are also much higher than previously reported values for particles this size.

As previously mentioned, it has been proposed that the blocking temperature of cobalt nanoparticles can be enhanced by coating the nanoparticles with an oxide layer. For example Skumryev et al. have reported synthesizing Co@CoO core-shell nanoparticles that exhibit a blocking temperature of 290 K. These authors suggest that an exchange bias between the core and outside shell leads to the enhancement. However, the blocking temperatures achieved by Skumryev et al. are near but generally still below room temperature. In contrast, exemplary embodiments of the present invention provide nanoparticle blocking temperatures of much higher value achieved by mixing a soft magnetic material with a non-magnetic material. The blocking temperatures of the present invention may be equal to or greater than 300 K, in the range of 300 K to at least 571 K, in the range of 400 K to at least 571 K, or in the range of 500 K to at least 571 K. The results not only show nanoparticles with a larger T_{B} but present a phase of Co₃C nanoparticles with ferromagnetic stability up to 571 K. This sets a new superparamagnetic limit at much higher temperatures than previous particles of similar size, including prior core shell nanoparticles.

### Example 2

In order to probe the microscopic origin of the observed large anisotropy, first principles density functional theory investigations were conducted. The investigations were directed to the magnetocrystalline anisotropy, Eₐₙᵢₛ, in the new phase of cobalt carbide, characterized by cobalt layers separated by carbon layers. Here, the Eₐₙᵢₛ was calculated by determining the contribution from spin-orbit coupling to the total energy by constraining the magnetic moment along various directions characterized by the spherical angles θ and φ. The total energy can be divided into two parts. The first is the direction-independent energy, and the second is the small angular-dependent variation of energy. The second part determines the so-called anisotropy energy, which can be expressed according to the following formula:

ΔE(θ,φ)=E(0,0)+V sin²(θ−θ₀)×{K+K′ cos [2(φ−φ₀)]}

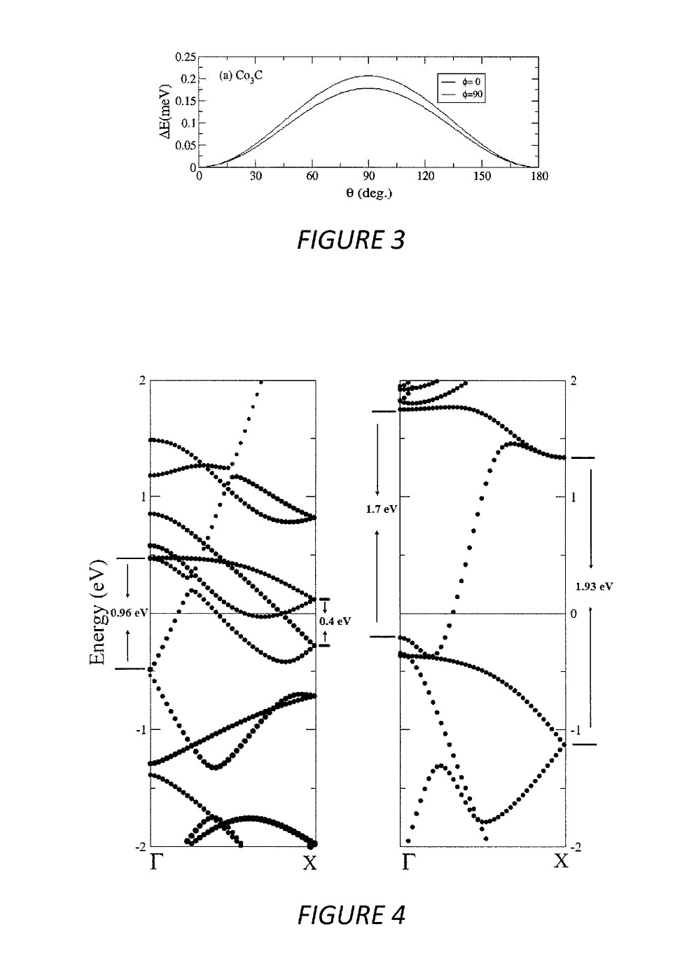

Here K and K′ are two magnetic anisotropy constants of a nanoparticle, and the spherical angles θ₀ and φ₀ correspond to the easy axis directed along a minimum of anisotropy energy. In order to determine K and K′, calculations of the ΔE(θ, φ) were first carried out by constraining the magnetic moment in various directions, until a local minimum of the total energy is reached. For Co₃C, an easy axis was identified along [001] direction with spherical angles θ₀=φ₀=0 (FIG. 3). As shown in FIG. 3, the ΔE (θ,φ) was calculated at different θ at constant φ=0° and φ=90°. The above equation for ΔE (θ,φ) was then fitted to the calculated energies to determine the anisotropy constants. The calculated K and K′ were determined to be 8.4×10⁵ J/m³ and −0.61×10⁵ J/m³ respectively. The fitting of the experimental data leads to an effective K_{eff} that does not involve variation over φ. Using the calculated constants according to the above equation, the theoretical K_{eff} lies between two values, minimum (K+K′) 7.8×10⁵ J/m³ at φ=0° and maximum (K−K′) 9.0×10⁵ J/m³ at φ=90°. The calculated values are in a good agreement with the experimental measurement of 7.5±1.0×10⁵ J/m³ noted above indicating that the primary contributor to the experimental anisotropy is the magnetocrystalline energy.

### Example 3

In order to further quantify how such a mixing leads to an increase in magnetic anisotropy energy (MAE), examination was made of the band structure and the electronic states with large d-character in the cobalt carbide materials. The MAE in transition metal systems is small. As has been previously shown, a second order perturbation calculation of the spin orbit interaction may provide a microscopic picture. Within the second order model, the MAE is determined by the matrix element of the spin orbit interaction between the occupied and the unoccupied states. The location of the occupied and unoccupied cobalt d-states close to Fermi energy were examined for three cases: pure bulk cobalt, cobalt carbide according to an exemplary embodiment of cobalt carbide nanoparticles having cobalt layers separated by carbon layers, and the structure of nanoparticles having cobalt layers without the carbon layers. FIG. 4 shows the energy bands along Γ to X for the compositional structure of cobalt carbide (Co₃C) according to the present invention and for the separated cobalt layers with the carbon removed (Co₃E). The states with larger d-component are shown by the dark dots. To further quantify the change in anisotropy, the energy difference between the states at the Γ and X points for the cobalt carbide nanoparticles and pure hexagonal cobalt (Table I) were examined. The separation into layers decreases the energy difference, thus increasing the anisotropy. Additionally, the mixing between the p-states of the carbon and d-states of the cobalt further reduces the energy difference thus contributing to comparatively giant anisotropy values.

TABLE I
MAE of bulk Co₃C and Co₃E (E = empty sphere) in
units of meV per formula. The zero energy is set as the
reference and the corresponding direction is the easy axis.

| | [100] | [010] | [001] | [110] | [111] |
|---|---|---|---|---|---|
| Co₃C | 0.178 | 0.206 | 0 | 0.191 | 0.128 |
| Co₃E | 0.109 | 0.016 | 0 | 0.062 | 0.042 |

### Example 4

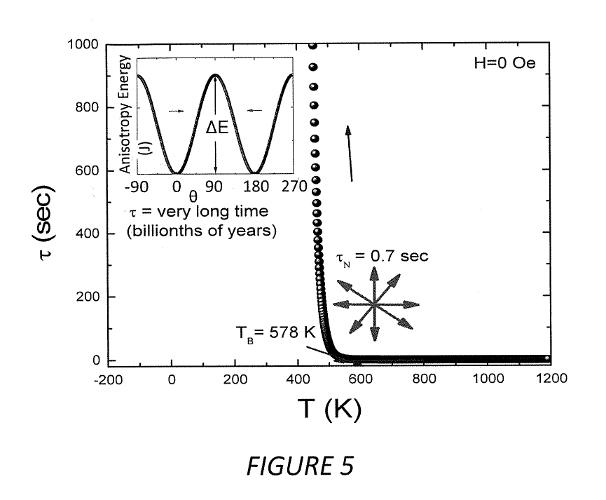

For applications of nanoparticles according to exemplary embodiments of the invention, it is worthwhile to investigate the fluctuation time between two magnetization directions known as Neel-relaxation time (τ_{N}). The Neel-relaxation time is related to the anisotropy energy according to the relationship, τ_{N}=τ₀ e^{KeffV/kBT}. Using the anisotropy values, the Neel-relaxation time was determined as a function of temperature, results being shown in FIG. 5. The inset shows the two minima of the anisotropy energy at θ=0° and 180° while the maximum anisotropy energy occurs when the magnetic moment is 90° to the easy axis. As shown in FIG. 5, at low temperature where the thermal energy is very small compared to the anisotropy energy, the Neel-relaxation time is very long (10⁹ years) revealing thermally stable magnetic order. As temperature is increased, the Neel-relaxation time remains relatively long until nearly 300 K, at which point it drops to 434 years (thermally stable magnetic order). Upon further raising the temperature, the Nee-relaxation drops significantly. At 578 K, near the blocking temperature T_{B} of 571 K, the fluctuation time drops to 0.7 s. At still greater temperatures, the magnetic moments fluctuate freely. As previously discussed, this occurs due to the increase of the thermal energy such that it becomes larger than the anisotropy energy. The magnetic order is then not thermally stable, and superparamagnetic (SPM) behavior dominates.

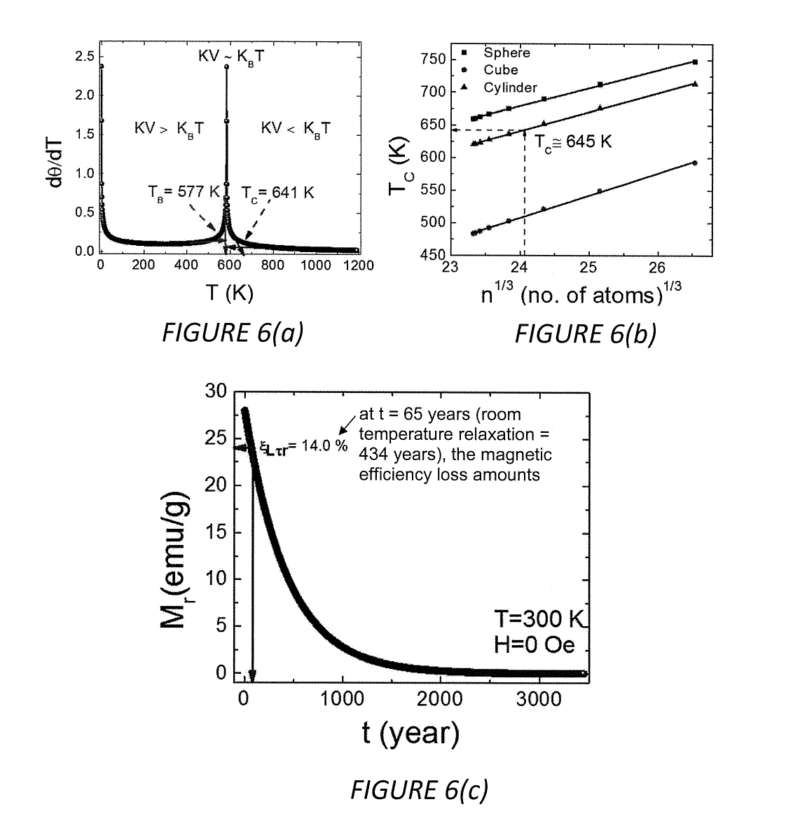

The observed anisotropy was also used to determine the rate of change of the magnetic moment direction (dθ/dt) as a function of temperature using the expression 25 k_{B}T=K_{eff}V Sin² θ. The results are shown in FIG. 6(a). From these, the blocking temperature T_{B} and the Curie temperature T_{C} (threshold between superparamagnetic behavior and paramagnetic behavior) are determined to be 577 K and 641 K, respectively. These values are in good agreement with the value determined from the H_{C} dependence of Temperature (FIG. 2(b)). At low temperature, K_{eff}V>k_{B}T, and the dθ/dt is very small indicating that the magnetic moment takes a long time to fluctuate from one direction to another direction. Once the temperature is close to T_{B}, the thermal energy is comparable to the anisotropy energy K_{eff}V≈k_{B}T and dθ/dt increases till it reaches the maximum value, and the superparamagnetic behavior dominates. Further increase in the temperature beyond T_{B} results in a decrease of the dθ/dt that becomes very small close to T_{C} at 641 K. Once T_{C} has been reached, the temperature effect on dθ/dt is negligible, and the magnetic moments take random directions and behave as paramagnetic.

Information regarding the shape of the particles can be determined from the T_{C} dependence on particle size by applying a cohesive energy model (FIG. 6(b)). As shown in the figure, the cohesive energy model provides a linear relationship between T_{C} of a particle and the cube root of a particle's total number of atoms. This linearity is observed with at least three different particle shapes, specifically sphere, cube, and cylinder as shown in FIG. 6(b). By comparing the experimentally determined T_{C} with the cohesive energy model shown, the cobalt carbide nanoparticles of the exemplary embodiment were indicated as being cylindrical shaped nanoparticles. This is consistent with the TEM analysis shown in FIG. 2(c). The cohesive energy model was also used to determine a calculated T_{C} based on the experimentally determined particle size. The calculated T_{C} was found to be around 645 K, which is in a good agreement with the experimental result based on M×H measurements.

One use of Co₃C nanoparticles as nanomagnets is in data storage applications, for which it is useful to determine magnetic efficiency loss. FIG. 6(c) shows the remnant magnetization (Mᵣ) dependence on time at zero magnetic fields (that is to say, in the absence of any external magnetic field) and at room temperature. The magnetic efficiency loss (ζ) at room temperature was found to be around 14% after 65 years of using the materials. This evidence supports the notion that a new door has been opened in data storage technology. Specifically, cobalt-carbide nanoparticles as presented herein and variations thereon in accordance with the teachings herein may be used a new materials in digital media storage and like applications.

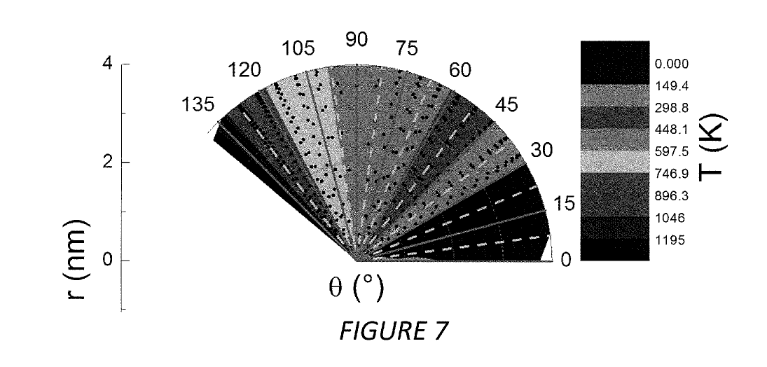

FIG. 7 shows the above findings on the effects of temperature and particle size on the direction and Neel-relaxation time of the magnetic moment condensed into a single simple 3D figure that represents the effect of temperature on the rotation of the magnetic moment. FIG. 7 indicates the change in the temperature range starting from the lower temperatures up to the very high temperatures. The effect of the thermal energy on change of the magnetic moment direction has been indicated from 0° to 135° resulting in a magnetic moment rotation image of the particle around its easy axis.

### Example 5

### Non Rare Earth High Performance Permanent Magnets Via Solution-Processed Assembly of Exchange Coupled Cobalt Carbide Nanoparticles

Rare earth permanent magnets have a high-impact on clean technology applications such as wind turbines and electric vehicle motors.¹ However, due to the restricted accessibility of imports of rare earth commodities to the United States rare earth permanent magnets are becoming increasingly expensive to manufacture. Alternative sources, such as rare earth free permanent magnets, with similar or enhanced energy products are becoming a subject of intense research.^{1, 2} Recently, mixed phase cobalt carbide nanoparticles were shown to possess enhanced magnetic properties, and due to the lack of rare earth elements, are very attractive for their use in clean-energy technologies.^{3, 4} However, to fully optimize the magnetic performance, a better understanding and control of the phases which compose the nanoparticles are still needed. Here we report the synthesis and characterization of pure phase Co₃C and Co₂C nanoparticles processed via a wet chemical technique. By studying the magnetic and thermal properties a detailed understanding of the formation mechanics and origin of the magnetic properties was elucidated. Determining the effects each phase has on the magnetic properties will lead to state-of-the-art permanent magnets by effectively enhancing their energy product to rival that of current technologies.

Permanent magnets are a key component in many energy related applications, where an increase in the magnetic energy density of the magnet, typically presented via the maximum energy product (BH)ₘₐₓ, increases the efficiency of the whole device (for example the volume-to-power ratio of an electric motor). Since the development of rare earth permanent magnets in the 1960's and 1970's there have been slight advances in the (BH)ₘₐₓ achieved by varying the synthetic processing and the ability to control the anisotropy. However, the discovery of novel materials with enhanced energy products has been limited.^{1, 2} The last major advance in non-rare earth permanent magnets dates back to the mid 1930's with the development of AlNiCo magnets and since then only slight changes in the (BH)ₘₐₓ have been reported.² The recent discovery of cobalt carbide nanoparticles has opened the door to a new class of non-rare earth permanent magnet materials that has potential to out perform AlNiCo and even that of the best rare earth permanent magnets. The synthesis of CoₓC nanoparticles is accomplished using a wet chemical technique, the polyol process, where a cobalt precursor salt is dissolved in a polyhydric alcohol (polyol) and heated to elevated temperatures (250-325° C.), near the boiling point of the solvent. At these elevated temperatures the polyol is at its highest reactivity and acts as solvent, capping agent, and reducing agent. Using this wet chemical approach, it is possible to tailor the magnetic properties to produce phase pure carbides thereby creating new high-energy product permanent magnet.

In a typical reaction, potassium hydroxide is dissolved in tetraethylene glycol (TEG) and heated to 275° C. Once the solution reaches temperature, the cobalt salt is added to the hot solution in ten increments over 20 minutes. In experiments using a singular addition of cobalt salt, the resulting particles were a composite of metallic cobalt and cobalt carbide phases. The incremental addition of the cobalt salt dramatically attenuates the growth steps allowing for the incorporation of carbon into the cobalt structure and results in the formation of pure carbide phase nanoparticles. The intricate control in the formation of phase pure Co₂C and Co₃C carbide is accomplished by varying the hydroxide concentration in the TEG solution. In a basic environment, the polyol will polymerize via a condensation reaction producing polyethylene glycol (PEG) of varying chain lengths. Increasing the hydroxide concentration increases the PEG chain length resulting in variations in the nucleation dynamics, which generates either form of the cobalt nuclei (α-Co or β-Co). At low hydroxide concentrations, the kinetic product α-Co (hexagonal closed packed, HCP) is formed upon nucleation, while higher concentrations form the thermodynamic product β-Co (face-centered cubic, FCC).^{5, 6} The initial structure of the nucleated cobalt then determines the final carbide phase formation; α-Co forms Co₃C while a mixture of α-Co and β-Co forms Co₂C. According to x-ray diffraction data and structural refinement, the calculated lattice parameters of the Co₂C sample are a=4.45 Å, b=4.37 Å, and c=2.90 Å (space group Pnnm) while the lattice parameters of Co₃C are a=5.02 Å, b=6.73 Å, and c=4.44 Å (space group Pnma) (XRD scans can be seen in the supporting information, S1). These lattice parameters are consistent with lattice parameters of bulk CoₓC particles.⁷

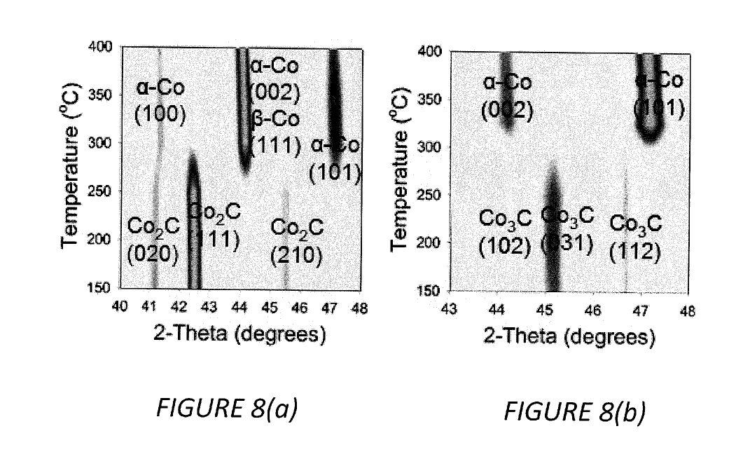

Elevated Temperature X-Ray Diffraction (ETXRD) further supported this correlation between the metallic structure and carbide phase (FIGS. 8(a),8(b)). Initially, as the temperature increases from 25° C. to 250° C., the carbon rich Co₂C decomposes creating a mixture of both α and β-Co. With the Co₃C, there is a reduced carbon content which results in a higher decomposition temperature at 325° C. Since there is less disruption to the carbide lattice only the α-Co is formed. While the relationship between metallic cobalt and carbide phases is interesting, it alone does very little to aid in the elucidation of the formation mechanism of the cobalt carbide system.

While few studies have addressed the formation of cobalt carbide nanoparticles, the formation of nickel carbides during nickel catalyzed Fisher-Tropsch synthesis has been extensively studied. Nickel crystallizes into similar metal and carbide allotropes as cobalt, so the formation of nickel carbides could be extended to the cobalt carbide system. At the elevated temperatures of the TEG, carbidization tends to occur according to a carbide cycle similar to ones reported with Fischer-Tropsch catalysts.⁸⁻¹² As the metallic cobalt nanoparticles nucleate, they catalyze the decomposition of the glycolate to form carbon on the surface of the nanoparticles. Due to the presence of these surface carbon atoms, surface diffusion occurs and alters the structure of the metallic cobalt nanoparticles. The formed carbide phase is dependent on the metallic cobalt phase upon nucleation, shown in the ETXRD data, and on the amount of surface carbon.¹³ Co₃C is formed from the carbon filling the hollow sites of the α-Co surface. For Co₂C, a p4g clock site reconstruction occurs on the α-Co (001) and β-Co (111) planes.^{9, 14} The increased carbon needed to induce a p4g surface reconstruction is a result of defects from the two phase particle system seen in the ETXRD study.¹³ While the carbon only diffuses into the surface layers, due to the attenuated growth rate resulting from the iterative additions, the carbon is continuously incorporated resulting in the complete conversion to the carbide. When the cobalt precursor is introduced instantaneously, the cobalt undergoes rapid growth. Since the carbon formation is slower than the cobalt growth, a multi-phase metallic/carbide particle is formed. However, by adding the cobalt precursor iteratively the growth occurs in stages, which allows for the complete incorporation of the carbon creating a pure carbide phase.

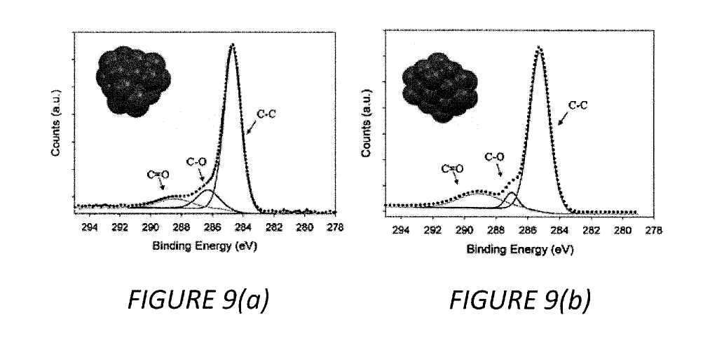

In previous reports on the synthesis of cobalt carbide, the carbon is identified as a graphitic carbon layer on the metallic nanoparticle.¹⁵⁻¹⁸ In this Example, X-ray photoelectron spectroscopy probed the surface of the carbide particles, in order to determine the structure of the surface carbons. The Carbon C 1 s scan of Co₃C and Co₂C shown in FIGS. 9(a),9(b) shows 3 carbon species: C═O at 288.5 eV, C—O at 286.3 eV, and C—C at 284.7 eV. The C—O and C═O species imply the presence of glycolate on the surface.¹⁹ Comparing the two phases, Co₂C has a higher ratio of C═O due to oxidation of the glycolate at higher pH. Interestingly, no carbide carbon was identified in the XPS scans of Co₃C or Co₂C.^{9, 11} Although, when looking at the space filled models in FIGS. 9(a), 9(b) (insets) the carbide carbon is present in low amounts on the surface of both Co₃C and Co₂C. A low concentration of surface carbon, coupled with the glycolate layer explains the inability to definitively identify the carbide carbon. Also XPS studies of deactivated cobalt based Fischer-Tropsch catalysts identify large amounts of fragmented hydrocarbons and adsorbed C—O species. The carbide carbon is only seen upon removal of the surface carbons by reduction in H₂.¹⁰

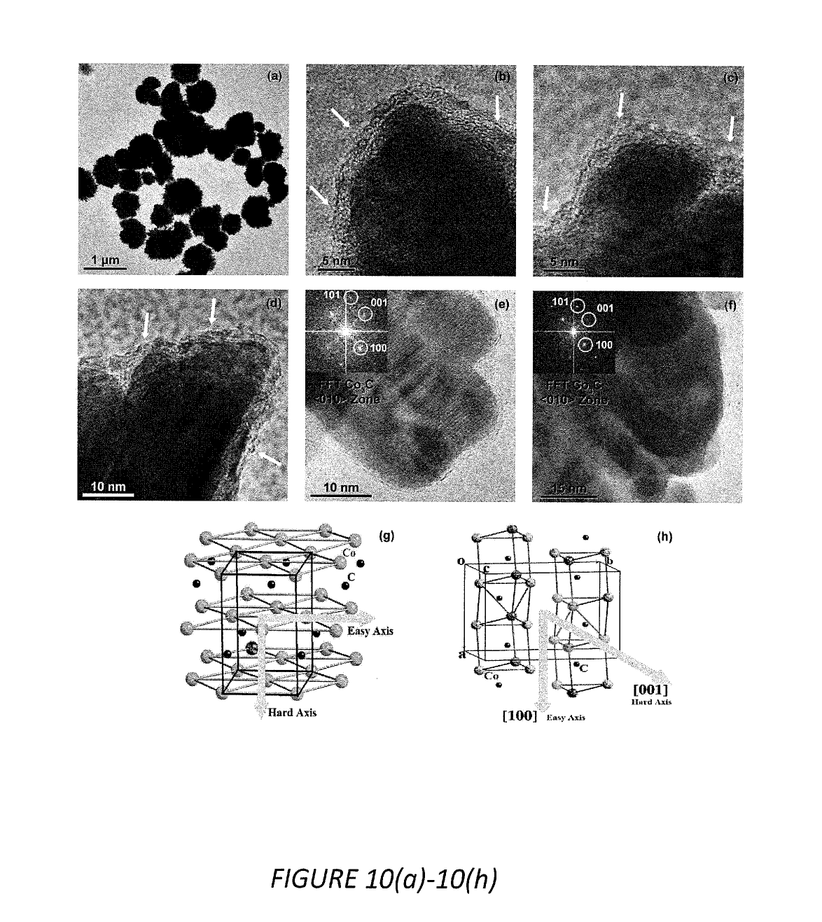

Synthesized Co₂C and Co₃C particles possess an average particle diameter around 300 nm (FIG. 10(a)). The Co₂C and Co₃C particles both possess a spherical shape with similar diameters (FIG. 10(b,c)). High-resolution TEM images of the particle surfaces reveal the presence of a glycolate layer and finer crystallites attached to larger agglomerates (FIG. 10(b-d)). This polycrystalline assembly was also confirmed from SAED and XRD Scherrer analysis. Fourier transforms of the images confirm Co₃C lattice parameters of a=5.05 A and c=4.48 A (FIG. 10(e,f)), commensurate with the values obtained from x-ray diffraction.

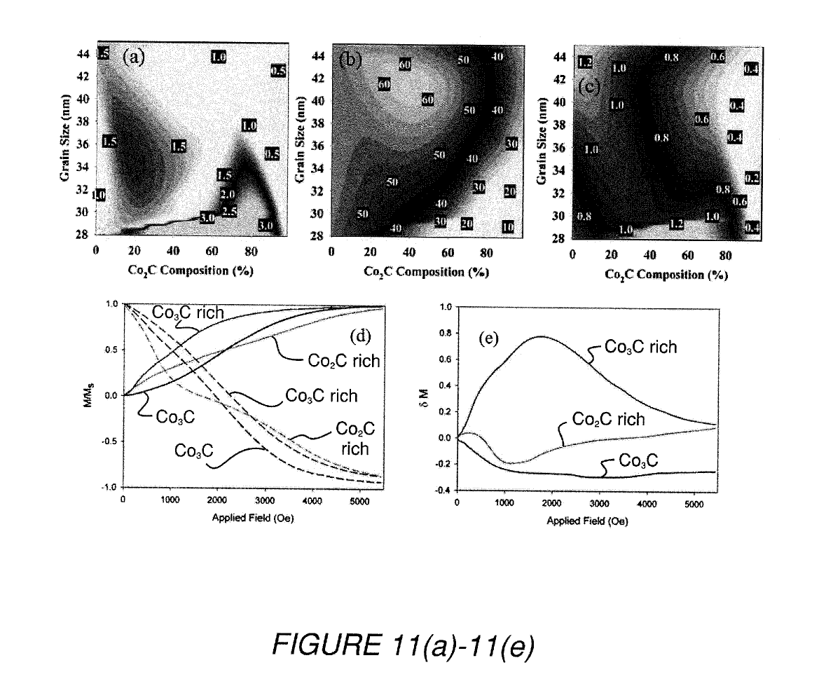

With the synthesis of pure phase Co₂C and Co₃C, the magnetic properties intrinsic to each phase can be accurately identified. Along with the two pure phases, CoₓC particles consisting of various phase ratios were also characterized. Single phase Co₂C was found to possess a low coercivity (450 Oe) and low magnetization (13 emu/g) while single phase Co₃C showed a high coercivity (1.6 kOe) and high saturation magnetization (55 emu/g) (FIG. 11(a,b)). Single phase Co₃C particles also possessed a higher (BH)ₘₐₓ (1.5 MGOe for Co₃C versus 0.1 MGOe for Co₂C) (FIG. 11(c)). BHₘₐₓ is calculated as the largest area rectangle formed from the product of magnetization and applied field in the second quadrant of a magnet's hysteresis curve. The high coercivity of the Co₃C can be attributed to magnetostatic interactions (FIG. 11(d)). At low field strengths the magnetic properties of the Co₃C system are dominated by dipole-dipole interactions from superparamagnetic components present. As Co₂C is doped into the system the dipole-dipole interactions are replaced by exchange coupling (FIG. 11(e)). As the Co₂C composition was increased to over 80% the degree of exchange coupling is reduced resulting in a decreased coercivity. Grain size also had an effect on (BH)ₘₐₓ values for the CoₓC particles, with lower grain sizes increasing exchange coupling.²⁰⁻²²

First principles theoretical studies examined the magnetic moment and the magneto-crystalline anisotropy energy (MAE) associated with the Co₂C and Co₃C phases. The exact theoretical methods can be found in the methods section. For pure β-Co, the calculated magnetic moment of 1.86μ_{b} per atom is close to the experimental moment of 1.81μ_{b}. In addition, the theoretical studies indicate that both Co₂C and Co₃C are metallic with moments of 1.0μ_{b}/atom for Co₂C and 1.65μ_{b}/atom for Co₃C, respectively. Our findings are in good agreement with the experimentally observed increase in magnetic moment for Co₃C over that of Co₂C. Bulk Co is a soft magnetic material (having a low coercivity, below 150 Oe) and our MAE calculations indeed indicate an easy axis along the [100] direction. For the [110] and [111] directions, the studies indicate a MAE per Co atom of 0.0092 and 0.0161 eV/atom respectively. For the Co₂C phase, the easy axis is along the [001] direction and the MAE/atom along [111] is 0.124 eV/Co₂C. The Co₃C phase, however, is unique as it has an [100] easy axis but the MAE increases to 0.15-0.19 meV/Co₃C along other directions. The uniaxial anisotropy of the compound contributes to the higher coercivity.

In summary, a modified polyol process was used to synthesize pure phase Co₂C and Co₃C nanoparticles. Through analysis of the carbide phases, a comprehensive understanding of the formation mechanism was developed. For the first time, control of CoₓC phase through hydroxide concentration, as well as other necessary synthetic parameters, is reported. Magnetic characterization and first principles theoretical studies of Co₃C and Co₂C identified the magnetic properties intrinsic to each phase. With the magnetic properties of each carbide phase, now known, it is anticipated that the energy product of CoₓC nanoparticles can be increased by tuning the size and composition of the individual motifs. The low cost of starting materials and high energy products will make the CoₓC nanoparticles an attractive replacement for current permanent magnets in many technology and industrial applications.

### Methods

In a typical experiment, tetraethylene glycol (TEG) was stirred, either magnetically or mechanically, and heated to 275° C. with varying amounts of KOH under distillation conditions. Once the solution reached the desired temperature, a total of 10 quantities of 2.0×10⁻⁴ moles of Co(Ac)₂.4H₂O were added individually at 2 minute intervals. After the final addition of cobalt precursor, the reaction was kept at temperature for 15 minutes and then cooled for an hour to room temperature. The solution was then centrifuged, rinsed several times, and magnetically separated. Rinsing was done first with ethanol several times, then a 5% HNO₃ in ethanol solution was used as the last rinse. The collected particles were then dried in a vacuum oven.

X-ray diffraction (XRD) scans were collected using a Panalytical X'Pert Pro MPD series diffractometer, with Cu Kα radiation (λ=0.154056 A) in θ-2θ geometry. For elevated temperature X-ray diffraction (ETXRD) an Anton Paar HTK-1200N high temperature camera was used coupled with a TCU-1000 temperature control unit. The ETXRD scans were collected under flowing N₂ atmosphere. Sample height was adjusted using a direct beam bisection method at each temperature to correct for thermal expansion. XRD analysis was carried out using X'Pert Highscore Plus software. For determination of grain size the XRD scans were first background corrected. They were then smoothed and FWHM for each peak was determined using the Profit algorithm. The Scherrer equation was then applied using the FWHM of the Co₂C (111) peak and the Co₃C (210) peak. Electron micrograph (TEM) examination was performed with a Zeiss Libra 120 operating at 120 kV and a JEOL 2100 LaB₆ operating at 200 kV. TEM samples were prepared by suspending the particles in ethanol and sonicating for five minutes. Small amounts were then pipetted onto ultrathin carbon TEM grids and the solvent was allowed to dry before imaging. Magnetic properties were determined using a Lakeshore VSM with a maximum applied field of 10 kiloOersted (kOe). Isothermal Remanance Magnetization (IRM) and Direct Current Demagnetization (DCD) plots were collected as a function of applied field. For an IRM plot, the magnetization was measured at zero field, then ramped to ΔH, and returned to zero field. The magnetization was then measured and repeated for increasing steps of ΔH. For the DCD plots, the sample was first saturated in negative field and returned to zero field. The magnetization was then measured as was explained for the IRM plot. AH for both IRM and DCD plots was 20 Oe. X-ray photoelectron spectroscopy (XPS) was performed on a Thermo Scientific ESCALAB 250 microprobe with a focused monochromatic Al Kα X-ray (1486.6 eV) source and a 180° hemispherical analyzer with a 6-element multichannel detector. The incident X-ray beam was 45° off normal to the sample while the X-ray photoelectron detector was normal to the sample. Angle resolved experiments were conducted at 4° increments from normal until peak intensities reached extinction. The samples were sputtered with gold clusters and binding energies were corrected to the Au 4 f peak at 83.95 eV. A large area magnetic lens with a 500 μm spot size in constant analyzer energy (CAE) mode was utilized with a pass energy of 20 eV. Five to twenty scans per region were collected based on sensitivity with step size of 0.100 eV. The powdered samples were pressed onto indium foil and secured to the sample holder using double-sided conductive carbon tape.

First Principles studies were carried out within a density functional framework using the Vienna Ab initio Simulation Package (VASP).^{23, 24} The projector-augmented wave method was used to model electron-ion interaction and the valence states of Co and C were described by [Ar] 3 d⁸ 4 s¹, and [He] 2 s² 2 p² electron configurations, respectively.²⁵ The exchange correlation contributions were incorporated using a hybrid functional B3LYP.²⁶ We also attempted generalized gradient functional proposed by Perdew, Burke, and Ernzerof in a GGA+U approach with a U value of 4.0 eV to find similar results.^{27, 28} A plane wave basis with an energy cutoff of 400 eV was used and a Mokhorst-Pack scheme of 5×5×5 division was used to generate the special k-points for constructing the charge density.²⁹ The magnetocrystalline anisotropy energy (MAE) was calculated using the contribution from spin-orbit coupling. For the Co₂C and Co₃C phases, the structures based on the x-ray diffraction were further optimized. Supplementary calculations were also carried out on bulk Co.

### REFERENCES FOR EXAMPLE 5

1 Sugimoto, S. Current status and recent topics of rare-earth permanent magnets. Journal of Physics D-Applied Physics 44, 11, doi:06400110.1088/0022-3727/44/6/064001.
2 Gutfleisch, O. et al. Magnetic Materials and Devices for the 21st Century: Stronger, Lighter, and More Energy Efficient. Adv. Mater. (Weinheim, Ger.) 23, 821-842, doi:10.1002/adma.201002180.
3 Harris, V. G. et al. High coercivity cobalt carbide nanoparticles processed via polyol reaction: a new permanent magnet material. Journal of Physics D-Applied Physics 43, 7, doi:16500310.1088/0022-3727/43/16/165003 (2010).
4 Zhang, Y. et al. Controlled synthesis and magnetic properties of hard magnetic CoxC (x=2, 3) nanocrystals. J. Magn. Magn. Mater. 323, 1495-1500 (2011).
5 Chakroune, N., Viau, G., Ricolleau, C., Fievet-Vincent, F. & Fievet, F. Cobalt-based anisotropic particles prepared by the polyol process. J. Mater. Chem. 13, 312-318, doi:10.1039/b209383a (2003).
6 Hinotsu, T., Jeyadevan, B., Chinnasamy, C. N., Shinoda, K. & Tohji, K. Size and structure control of magnetic nanoparticles by using a modified polyol process. J. Appl. Phys. 95, 7477-7479, doi:10.1063/1.1688534 (2004).
7 Pearson, W. B. Handbook of Lattice Spacings and Structures of Metals. Vol. 2 (Pergamon Press, 1967).
8 Cheng, J. et al. Density Functional Theory Study of Iron and Cobalt Carbides for Fischer-Tropsch Synthesis. J. Phys. Chem. C 114, 1085-1093, doi:10.1021/jp908482q.
9 Tan, K. F., Xu, J., Chang, J., Borgna, A. & Saeys, M. Carbon deposition on Co catalysts during Fischer-Tropsch synthesis: A computational and experimental study. J. Catal. 274, 121-129, doi:10.1016/j.jcat.2010.06.008.
10 Beitel, G. A., de Groot, C. P. M., Oosterbeek, H. & Wilson, J. H. A Combined in-Situ PM-RAIRS and Kinetic Study of Single-Crystal Cobalt Catalysts under Synthesis Gas at Pressures up to 300 mbar. The Journal of Physical Chemistry B 101, 4035-4043 (1997).
11 Xiong, J. et al. The formation of Co2C species in activated carbon supported cobalt-based catalysts and its impact on Fischer-Tropsch reaction. Catal. Lett. 102, 265-269 (2005).
12 Bezemer, G. L. et al. Cobalt particle size effects in the Fischer-Tropsch reaction studied with carbon nanofiber supported catalysts. J. Am. Chem. Soc. 128, 3956-3964, doi:10.1021/ja058282w (2006).
13 Stolbov, S., Hong, S. Y., Kara, A. & Rahman, T. S. Origin of the C-induced p4g reconstruction of Ni(001). Physical Review B 72, doi:155423 10.1103/PhysRevB.72.155423 (2005).
14 Lee, B. W., Alsenz, R., Ignatiev, A. & Van Hove, M. A. Surface structures of the two allotropic phases of cobalt. Physical Review B 17, 1510 (1978).
15 Wiltner, A. & Linsmeier, C. Formation of endothermic carbides on iron and nickel. Physica Status Solidi a-Applied Research 201, 881-887, doi:10.1002/pssa.200304362 (2004).
16 Pola, J. et al. IR laser-induced formation of amorphous Co—C films with crystalline Co, Co2C and Co3C nanograins in a graphitic shell. Journal of Photochemistry and Photobiology a-Chemistry 210, 153-161, doi:10.1016/j.jphotochem.2010.01.003.
17 Zaikovskii, V. I., Chesnokov, V. V. & Buyanov, R. A. State of disperse alloy particles catalyzing hydrocarbon decomposition by the carbide cycle mechanism: TEM and EDX studies of the Cu—Ni/Al2O3 and Cu—Co/Al2O3 catalysts. Kinetics and Catalysis 47, 603-609, doi:10.1134/s0023158406040173 (2006).
18 Huo, J. P., Song, H. H., Chen, X. H., Zhao, S. Q. & Xu, C. M. Structural transformation of carbon-encapsulated iron nanoparticles during heat treatment at 1000 degrees C. Mater. Chem. Phys. 101, 221-227, doi:10.1016/j.matchemphys.2005.12.048 (2007).
19 Carroll, K. J., Reveles, J. U., Shultz, M. D., Khanna, S. N. & Carpenter, E. E. Preparation of Elemental Cu and Ni Nanoparticles by the Polyol Method: An Experimental and Theoretical Approach. J. Phys. Chem. C 115, 2656-2664, doi:10.1021/jp1104196 (2011).
20 Fischer, R., Schrefl, T., Kronmüller, H. & Fidler, J. Grain-size dependence of remanence and coercive field of isotropic nanocrystalline composite permanent magnets. J. Magn. Magn. Mater. 153, 35-49 (1996).
21 Billoni, O. V., Urreta, S. E., Fabietti, L. M. & Bertorello, H. R. Dependence of the coercivity on the grain size in a FeNdB+[alpha]Fe nanocrystalline composite with enhanced remanence. J. Magn. Magn. Mater. 187, 371-380 (1998).
22 Feng, W. C., Gao, R. W., Li, W., Han, G. B. & Sun, Y. Dependence of coercivity on phase distribution and grain size in nanocomposite Nd2Fe14B/alpha-Fe magnets. Chinese Physics 14, 1649-1652 (2005).
23 Kresse, G. & Furthmuller, J. Efficient iterative schemes for ab initio total-energy calculations using a plane-wave basis set. Physical Review B 54, 11169-11186 (1996).
24 Kresse, G. & Hafner, J. Norm-conserving and ultrasoft pseudopotentials for first-row and transition-elements. Journal of Physics-Condensed Matter 6, 8245-8257 (1994).
25 Kresse, G. & Joubert, D. From ultrasoft pseudopotentials to the projector augmented-wave method. Physical Review B 59, 1758-1775 (1999).
26 Becke, A. D. A new mixing of hartree-fock and local density-functional theories. J. Chem. Phys. 98, 1372-1377 (1993).
27 Dudarev, S. L., Botton, G. A., Savrasov, S. Y., Humphreys, C. J. & Sutton, A. P. Electron-energy-loss spectra and the structural stability of nickel oxide: An LSDA+U study. Physical Review B 57, 1505-1509 (1998).
28 Perdew, J. P., Burke, K. & Ernzerhof, M. Generalized gradient approximation made simple. Phys. Rev. Lett. 77, 3865-3868 (1996).
29 Monkhorst, H. J. & Pack, J. D. Special points for brillouin-zone integrations. Physical Review B 13, 5188-5192 (1976).
30 Wohlfarth, E. P. Relations between Different Modes of Acquisition of the Remanent Magnetization of Ferromagnetic Particles. J. Appl. Phys. 29, 595-596 (1958).

### Example 6

### Extension of Findings with Co to Other Group II Elements

The results described in Example 6 can be extrapolated to alloy materials made with other metals. For example, calculations involving Fe₃C indicate that the Fe sites have a moment of 2.58μ_{B}/atom and an energy difference of 0.815 meV/Fe₃C between the easy axis and hard axis. The corresponding calculated moment per atom in bulk BCC Fe is 2.76μ_{B}/atom with an energy difference of 0.0092 meV/atom. These calculations indicate that by substituting iron for cobalt, the energy product of the resulting magnetic material could increase by 50% over the cobalt.

When complete information regarding a chemical mechanism is lacking, energy calculations can be used to successfully predict additional metals that might be employed in a synthesis method. For example, using the deMon2k software, a variational fitting of the Coulomb potential is employed to avoid the calculation of four-center electron repulsion integrals using the GEN-A2 function set. The exchange-correlation potential is calculated via a numerical integration from the orbital density. All electrons are treated explicitly using the double-ξ valence plus polarization basis sets optimized for generalized gradient functionals. A quasi-Newton method in delocalized internal coordinates is used for the geometry optimization. Several initial configurations are considered and the geometries fully optimized without any symmetry constraints to determine the ground state of the different species that are further ascertained through frequency analysis.

The cobalt carbide nanomagnet described in Example 5 is a novel material that demonstrates properties not found in the original bulk materials. When initial calculations are performed on the system, the particular origins of the high coercivity become clear. First principles theoretical studies examined the magnetic moment and the magneto-crystalline anisotropy of the Co₂C and Co₃C phases. The studies were conducted within a density functional framework using the Vienna Ab initio Simulation Package (VASP) and the exchange correlation contributions were incorporated using a hybrid functional B3LYP and a GGA+U approach. The magnetocrystalline anisotropy energy (MAE) was calculated using the contribution from spin-orbit coupling. For the Co₂C and Co₃C phases, the structures based on the x-ray diffraction were further optimized. Supplementary calculations were also carried out on bulk Co.

For pure β-Co, the calculated magnetic moment of 1.86 μb per atom is close to the experimental moment of 1.81 μb per atom. Theoretical studies indicate that both Co₂C and Co₃C are metallic; however, the bulk Co moment is reduced to 1.0 μb/atom for the Co₂C and 1.65 μb/atom for Co₃C phase, respectively. Our findings therefore agree with the experimental observation of the increase in magnetic moment from Co₂C to Co₃C phase. Bulk Co is a soft magnetic material and our MAE calculations indeed indicate an easy axis along [100] direction. For the [110] and [111] directions, the studies indicate a MAE per Co atom of 0.0092 and 0.0161 eV/atom, respectively. For the Co₂C phase, the easy axis is along the [001] direction and the MAE/atom along [111] is 0.124 eV/Co2C. The Co₃C phase, however, is unique. It has a [100] easy axis but the MAE increases to 0.15-0.19 meV/Co3C along other directions. The uniaxial anisotropy of the compound contributes to higher coercivity. Experimental results are consistent with these theoretical findings.

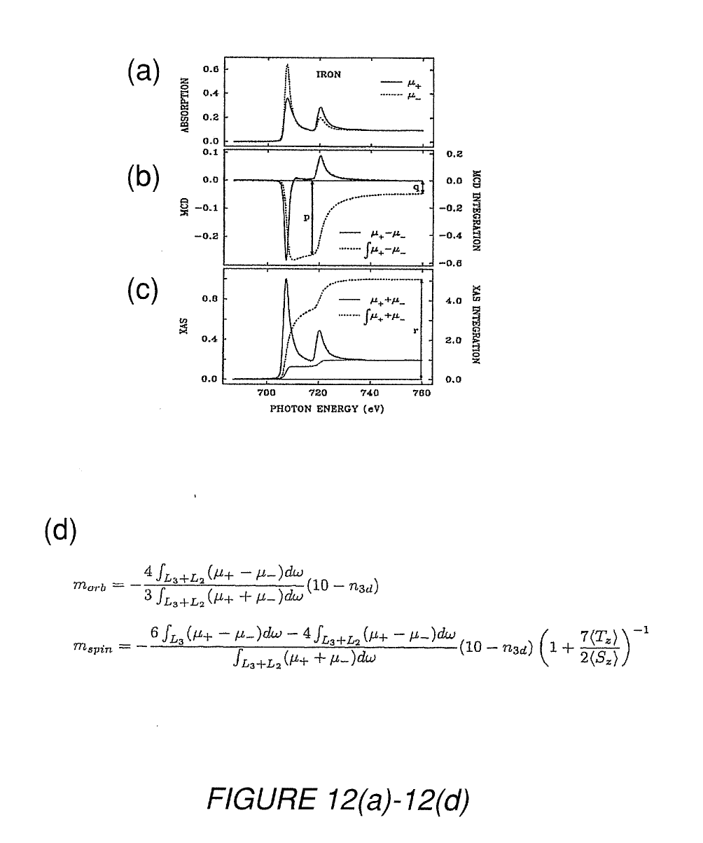

In examining complex magnetic systems, which may contain multiple magnetic elements often in multiple lattice sites or confined to specific layers in a multilayer structure, magnetization measurements that average over the whole sample can provide an incomplete picture of the interactions governing macroscopic properties. A local probe that includes magnetic sensitivity can illuminate the interactions among the various constituents; x-ray magnetic circular dichroism (XMCD) is the most well-established spectroscopic technique that can separately map out the contributions of individual components in complex magnets. XMCD provides a number of important capabilities that benefit the search for enhanced B—H product carbides: (a) element-specificity, (b) separation of spin and orbital moments, (c) sensitivity to crystal field, (d) depth-selectivity, and (e) element-specific magnetometry. In its simplest form, XMCD can be described as the differential absorption of circularly polarized x-rays based on the alignment of the helicity of the circularly polarized photons and the magnetization of the sample. XMCD consists of pairs of XAS scans with the photon helicity and the sample magnetization in an aligned or antialigned configuration (most commonly accomplished with the sample magnetized to saturation in the positive and negative directions). The XCMD spectrum is the difference between these two scans. The separation of the spin and orbital moments arises from the spin-orbit coupling that splits the atomic core levels with non-zero orbital angular momentum (i.e. l≠0, where l is the orbital angular momentum quantum number). In the case of 2nd row transition metal (TM) L edges, this results in the spin-orbit split 2p3/2 (total angular momentum J=L+S=1+½=3/2) and 2p½ (J=L−S=1−½=½). Under the dipole approximation, which is typically used to examine XAS spectra, sumrule analyses indicate that by taking properly weighted sums or differences (more accurately, integrals of these quantities) of the XMCD spectra, (FIG. 12) the orbital (morb) and spin (mspin) moments of specific elements can be measured separately, as shown in FIG. 12(d).

In the expressions shown in FIG. 12(d), the integration limits (e.g. L3, L2 etc.) refer to the L3 (2p3/2) and L2 (2p1/2) core levels, μ+ (μ−) are the XAS spectra collected in the aligned (antialigned) configurations, and the quantity (1−n3d) counts the number of unoccupied 3d states (i.e. the number of 3d holes) in the local density of states (LDOS) around the transition metal ion. The last item in brackets in the spin moment expression is a small higher-pole correction to the spin moment and is typically ignored.

In analyzing the XMCD spectra, the greatest source of uncertainty is the determination of the 3d-hole population, as both morb and mspin are directly proportional to this quantity. Comparison with first principles calculations undertaken as part of this proposal will therefore be extremely useful, as this is a quantity that comes directly from such calculations.

As mentioned above, XAS is a local probe and it is sensitive to the chemical environment surrounding the absorbing atom. The resulting spectra therefore reflect the local density of states around an atom as well as the crystal field. Multiplet effects in the spectra serve as fingerprints of both the oxidation state of cations in a sample as well as the local crystal field environment. This will be of great use in monitoring the local chemical environment around the Co atoms and other elements that are incorporated into the samples. The depth selectivity of XAS and XMCD arises from the different escape depths of the particles carrying the spectroscopic signal from the sample. Total electron yield (TEY) is the simplest method and provides a depth sensitivity of 4-10 nm from the sample surface (note this may be the surface of a nanoparticle). Partial electron yield (PEY) uses electric fields to eliminate the signal from the low energy secondary electrons, hence driving the electron mean free path down to reduce the depth sensitivity to ˜0.5-1 nm. Fluorescence yield (FY) is a photon-in/photon-out technique, and hence provides much greater depth sensitivity. In the case of soft xrays, FY signals can probe buried layers dozens of nm below the surface. Therefore, by combining different measurement techniques (TEY, PEY, FY) from the same sample, a depth profile of the sample magnetometry is developed, which can in turn reveal differences in the surface magnetization and chemical composition. Such studies are used to devise strategies for successful compaction and scale-up of the discovered high B—H product materials.

Finally, field-dependence of the coupling between different magnetic elements can be revealed through element-specific magnetic hysteresis (ESMH) loops, measured with XMCD. EMSH is measured by tuning the x-ray energy to a core level of choice (e.g. the Co and/or Fe L3 edge), fixing the helicity of the x-ray beam, and recording the signal as a function of applied magnetic field. In composite systems, such measurements reveal if the switching is associated with a single phase or a single element. Also, by combining the field sweeps with the different detection methods previously mentioned, differences in the magnetometry between surface and bulk are revealed. Lastly, EMSH measurements are a key tool in investigating interfacial phenomena such as exchange bias, which are also valuable.

These calculations led us to the synthesis of several different iron carbide alloys, namely Fe₃C, Fe₅C₂, and Fe₇C₃. The compounds have enhanced coercivities (over 100× that of bulk FexC) resulting from the increased anisotropy. Currently, the Fe₃C materials that have been made are larger nanoparticles. As the size is reduced the coercivities will increase as they did with the cobalt system.

### Example 7

### Further Developments

Permanent magnets, specifically those containing rare earth metals, are an indispensible component of many applications in electric, electronics, communications, and automobile industries. The emergence of green technology markets such as hybrid/electric vehicles (PHEVs and EVs), direct drive wind turbine power systems, and energy storage systems (eg: flywheels) has created an increased demand for permanent magnets.

Further developments regarding the synthesis of the permanent magnet cobalt carbide system allows the synthesis of next generation high energy permanent magnets with energy products exceeding 45 MGOe, providing a new class of permanent magnet materials that are superior to existing magnetic materials at a substantially reduced cost.

A wide size distribution of crystallites in the nanocluster is sometimes observed. The presence of smaller crystallites in the system can give rise to a superparamagnetic impurity that reduces the squareness of the magnetic hysteresis curve. The size and distribution of the nanoparticles is controlled thereby increasing the overall remanence magnetization. In addition, controlling the size allows the creation of specific sized particles that are used in exchanged coupled nanocomposites. Particle or cluster size is controlled, for example, by reaction time, concentration of the precursors, and pH. Surfactants may also be utilized. Polyvinylpyrrolidone (PVP) is a surfactant commonly used in the polyol process to control particle size. Due to cobalt's preference for Co—N bonding rather than Co—O, PVP is preferentially absorbed on the particle surface and slows growth. The ratio of surfactant to metal concentration helps to determine the size of the particle, as does the molecular weight of the PVP. By tailoring the size of the particles, the coercivity of the system is increased. Surface effects result in an increase in coercivity to a maximum as size is reduced, providing e.g. an increase of 50% of the overall energy product. Although Other surfactants may be employed, e.g. cetyltrimethylammonium bromide, nonylphenyl polyethoxylates, and polyvinyl alcohols are used to control the particle size in the polyol process.

Shape anisotropy is introduced to carbide particles to increase the coercivity. In Co nanoparticle systems, this has resulted in an 800% increase in the overall coercivity of the system is approximately doubled, resulting in dramatically increased coercivities and energy products, leading to an energy product increase of above 20 MGOe. In the polyol process, shapes are created by four different methods: solvent, surfactants, catalysts, and reaction conditions. Complex shapes of copper nanoparticles have been made by varying the solvent used in the system. When the surfactants absorb on the surface of the growing nuclei, they do not affect the growth along all crystal planes in the same way. This can lead to the formation of rods, cubes, or ellipsoids. Shape is also controlled by using catalysts such as ruthenium chloride. Reaction conditions also can affect the size of growing nanoparticles. By varying the temperature, precursors, stirring method, and the pH of the reaction solution, particles with a variety of lengths and aspect ratios are formed.

In addition, (BH)max is increased through exchange coupling. In exchange coupling, the high magnetization of soft magnetic materials is coupled with the high coercivity of hard magnetic materials. The development of these exchanged coupled magnets can be through two very different routes: Core-shell morphology and nanocomposites. Core-shell nanoparticles can be synthesized by controlling the precipitation and growth steps of the reaction. For example, one metal is allowed to nucleate and grow before another metal is introduced. The thermodynamics of crystal growth favor the growth of a particle over the nucleation of another. As a result, the second metal grows on the first, creating the core-shell particle. Various soft magnetic metals and alloys as used as cores for a cobalt shell since hard magnetic carbides are more air stable and provide greater protection of the soft metallic cores. Alternate materials are used to replace the cobalt carbides. The second major method for achieving exchange coupling is through mixing of nanocomposite materials. For example, hard and soft magnetic materials are created separately and then physically mixed together and compacted. Two different polyol reactions are conducted together and the products are mixed immediately prior to particle clean-up, providing uniformity. By mixing two different sizes of particles, a greater packing density is attained, further improving the overall magnet properties. Particle size is determined using Hume-Rothery rules, which are based on the simple geometry of stacked rigid spheres of different sizes. Optimal packing occurs when the ratio of the radii between the spheres is 0.414 to 0.732. So for example, 20 nm iron cobalt nanoparticles are used with 30 nm cobalt carbide nanoparticles. This doubles the overall magnetization, resulting in a 3× increase in the energy product.

An important step in the treatment of compacts is sintering. The goal of sintering is to further improve the density of the compacts. Sintering also affects, either positively or negatively, the sample microstructure. Because sintering temperatures and times affect both density and morphology, the interplay of the two is carefully controlled. In the case of crystallographic texture, optimal density is achieved without inducing secondary recrystallization, since recrystallization could result in the loss of much of the crystallographic texture and hence lower the Br and consequently the (BH)max.

For batch scale-up, the laboratory synthesis is essentially carried out in larger reactors and the mixing, heating, and material handling are optimized. For large scale syntheses, overhead stirring is viable. Consequently, the effects of stirring on the morphology and magnetic properties of the system are evaluated. In the laboratory syntheses, magnetic stirring resulted in more blade-like particles while overhead stirring leads to more spherical particles. In both cases, the magnetic properties are the same. In larger reaction volumes, where the mixing is less efficient, diffusion is a concern; therefore different stirring conditions are evaluated for impact on the product. Because mixing is less efficient in large batches, material handling is also an issue and is considered. Heating is also controlled. On a 100 ml scale, the heating rate is controlled, e.g. at 15° C./minute. The process is adjusted for larger volumes, e.g. 5 or more liters. Microwave heating may be used. Flow processing, described in Example 8 below, is used to produce the material for production of magnets.

Theoretical calculations (e.g in situ x-ray adsorption spectroscopy (XAS), x-ray magnetic circular dichrosim (XMCD), mechanistic modeling, etc. are used to identify additional substitutional metals or intercalates that further increase the energy product. In addition, other compositions are identified as substitutions for Co and/or C and are synthesized. For example, iron is an alternate to cobalt. Using the higher magnetization of iron and the higher anisotropy energy of iron carbide, the energy product is increased as much as 500%, thereby increasing the energy product of above 40 MGOe. Pressing (compaction) and sintering increase the energy product by over 100% due in part to alignment of the magnetic moment. Through this series of improvements, the 45 MGOe goal is attained.

The technology is scaled up to industrial scales of powder processing, engineering of finish components, for the design, fraction, and testing of PM motor prototypes.

### Example 8

### Flow Through Reactor for the Synthesis of Magnetic Nanoparticles

Wet chemical synthesis of nanoparticles provides the greatest control over the morphology and composition of the products. Additionally, wet chemical synthesis is the only method that can yield core-shell nanoparticles which have tremendous application in catalytic reactions as well as electronic and biomedical applications.

With the growth in potential nanotechnology applications, different methods are being developed to synthesize nanoparticles or nanocatalysts. These nanoparticle synthesis methods are typically conducted as bench-scale batch reactions as proof-of-concept studies. Subsequent practical applications of nanoparticles necessitate the production of large quantities of uniformly functional particles. Thus a critical step in bringing such applications to main stream use is to increase throughput of nanoparticle synthesis while maintaining uniform functional properties.

To that end, we have designed and fabricated a microfluidic reactor for the synthesis of the nanoparticles described herein. The microfluidic reactor module was attached to a T-mixer with peristaltic pumps for the two feed streams. Silver nanopartices were synthesized as follows: a first stream was a mixture of 0.01 M silver nitrate and a second stream was a mixture of 2 M formaldehyde and sodium hydroxide. The two streams were mixed to maintain a 1:1 ratio of NaOH to AgNO₃. At a flow rate of 1.5 ml/min, the mean residence time in the reactor was approximately 4 minutes. Synthesized nano-silver particles were collected, washed and dried in a vacuum over at 37° C. overnight. The nanoparticles were analyzed using X-ray diffraction (XRD). The results showed that a wide range of size distributions of the nanoparticles was produced. To narrow the range, the mixing rate of the two streams is adjusted to provide a more uniform flow.

Additional nanoparticles are synthesized using continuous flow, including:

1) Fe₂O₃ and Fe₃O₄ nanoparticles, by using a metal salt solution of FeCl₂ and a base solution of NH₄OH with the surfactant system of aerosol bis(2-ethylhexyl) sodium sulfosuccinate (AOT)-isooctane.
2) CdS, using CdCl₂ as the cation solution and Na₂S are the anion solution with the surfactant system of AOT-isooctane.
3) Fe, using FeCl₂ as the cation solution and NaBH₄ as the reducing agent with the surfactant system nonyl phenol polyethoxylate-toluene.
4) Cobalt carbides, using CoCl₂ in tetraethylene glycol and NaOH in tetraethylene glycol.

### Example 9

### Ethanol Assisted Reduction and Nucleation of Ferromagnetic Co and Ni Nanocrystalline Particles

The solvent assisted reduction of late row 3d transition metals commonly employs solvent systems such as polyhydric alcohols or long chain alkylamines. In this report, we investigate the ability of a primary alcohol to reduce Co and Ni. Using a solvothermal approach, we demonstrate the reduction and nucleation of Co and Ni particles using ethanol as a solvent and reducing agent. Nucleated Co and Ni particles were polycrystalline with grain sizes of 25 and 34 nm, respectively. Ni particles were spherical in shape with average diameters of 300 nm, while the Co particles agglomerated heavily, into micron scale secondary particles. High resolution transmission electron microscopy imaging revealed the presence of stacking faults in the synthesized Co particles. The highly crystalline Ni and Co particles display room temperature ferromagnetism, possessing magnetic saturation values comparable to bulk Ni and Co. The results presented provide a foundation for the use of primary alcohols as an effective and cost efficient reducing agent for other transition metal particle systems.

Nanocrystalline magnetic materials draw considerable attention due to their interesting magnetic properties when compared to bulk materials.¹ Through proper control of a particle's crystal grain size and shape, magnetic properties can be manipulated for the particle system. A common method used to accomplish control of a particle's shape and size is a solution based synthetic route.¹ The solution based synthesis of magnetic nanoparticles commonly employs a reducing agent and heat to initiate a redox reaction, where the reducing agent can be in the form of a reactant, like borohydride based salts and hydrazine, or in the form of a solvent with sufficient reducing ability.¹

The most common solvents used for the solvent assisted reduction of magnetic nanoparticles are polyhydric alcohols (polyols) and long chain alkylamines.¹⁻⁷ Fievet et al. first demonstrated the ability to reduce of Ni(OH)₂ and Co(OH)₂ in ethylene glycol to form magnetic particles in 1988.⁸ Since this initial discovery, numerous ferromagnetic particle systems in a wide range of polyhydric alcohols have been investigated. While numerous reports demonstrate the solvent assisted reduction and formation of Ni and Co magnetic particles in polyols and alkylamines, no reports of using lower boiling point primary alcohols as a reducing agent to synthesize Ni and Co particles exist.^{2, 3, 5-7, 9} The use of lower boiling point solvents can increase cost efficiency by lowering energy consumption leading to a more industrially viable process. In this study, we report the synthesis of Ni and Co particles using ethanol as a solvent and reducing agent.

While the synthesis of Ni and Co nanoparticles using ethanol as a reducing agent has not been reported, the reduction of Ni and Co by ethanol has been observed during the catalytic process of Ethanol Steam Reforming (ESR) and when using primary alcohols under supercritical conditions.¹⁰⁻¹² To investigate the viability of ethanol as a reducing agent for the synthesis of Ni and Co particles under sub-critical conditions, 1 mmol of nickel or cobalt acetate was dissolved in 25 mL of a 95% ethanol solution. Along with the nickel and cobalt precursors 6×10⁻³ mmol of K₂PtCl₄ was added as a nucleating agent. The solution was then sonicated to dissolve the metal salts, sealed in a 50 mL capacity autoclave and heated to 200° C. for 2.5 hours. The vessels were then cooled to room temperature and the resulting particles were rinsed with ethanol and magnetically separated.

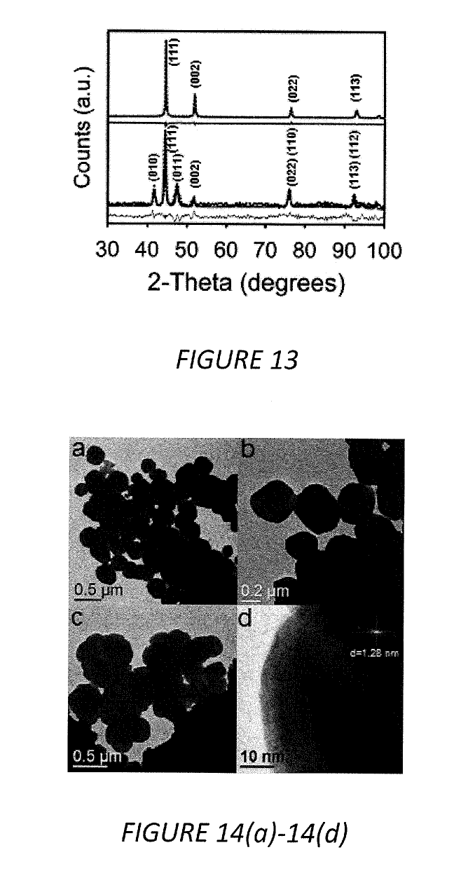

The heterogenenous nucleation of Ni particles formed pure phase face centered cubic (FCC) Ni with an average crystal grain size of 34 nm. Reduction of cobalt acetate nucleated particles comprised of 54% hexagonal close packed Co (HCP) and 46% FCC—Co crystal phases. A two phase system consisting of HCP—Co and FCC—Co is commonly observed in the solution based of Co particles, and can be attributed to the low energy barrier required for stacking fault formation to occur between the Co allotropic structures.^{3, 9, 13, 14} The formation of stacking faults commonly introduces disorder along the HCP (011) plane, which can be witnessed as broadening of the HCP (011) peak when compared to the other HCP peaks for the as prepared Co particles in FIG. 13.³ The grain sizes for the HCP and FCC—Co phases were calculated to be 25 nm and 28 nm, respectively. Crystal structure refinement for the FCC—Ni, FCC—Co, and HCP—Co revealed lattice constants slightly increased over bulk values.¹⁵ Inductively Coupled Plasma-Optical Emission Spectroscopy (ICP-OES) revealed Pt molar percentages relative to Ni and Co of 0.22% and 0.94%, respectively. The results from crystal structural refinement and ICP-OES indicate alloy formation to occur between Ni or Co and Pt atoms employed as a nucleating agent.

TABLE II
Refined Lattice Parameters for FCC Ni, FCC-Co, and HCP-Co
phases (Bulk values below refined values in brackets)

| | Space | Lattice Parameters (A) | | | Mass | Molar |
|---|---|---|---|---|---|---|
| Phase | Group | a | b | c | % Pt^{a} | % Pt^{a} |
| FCC-Ni | Fm-3m | 3.526 | — | — | 0.74 | 0.22 |
| | | [3.52] | | | | |
| FCC-CO | Fm-3m | 3.546 | — | — | 3.12 | 0.94 |
| | | [3.544] | | | | |
| HCP-Co | P63mmc | 2.514 | — | 4.105 | 3.12 | 0.94 |
| | | [2.507] | | [4.070] | | |

^{a}Pt percentages measured using ICP-OES

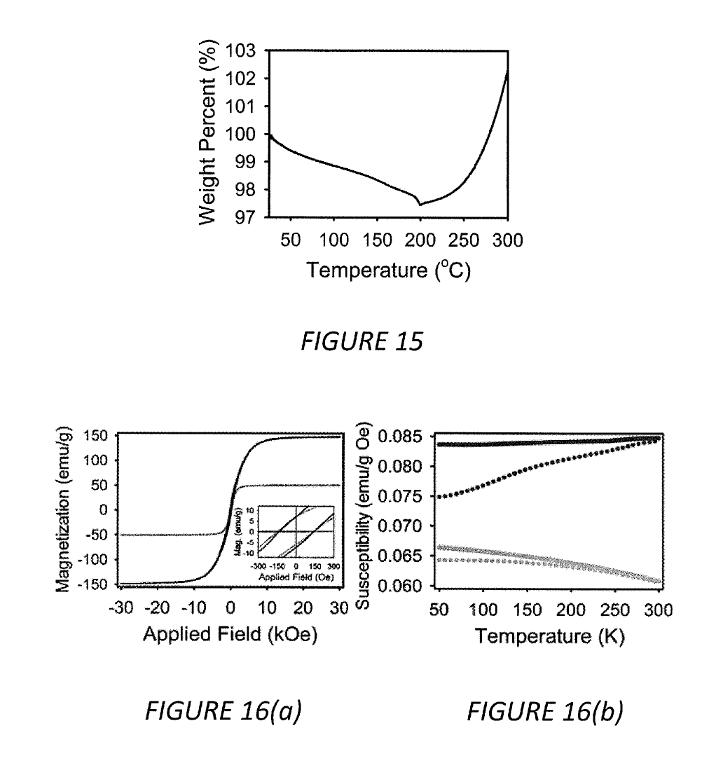

Transmission Electron Microscopy (TEM) images of the synthesized Ni particles revealed the particles to be spherical in shape with an average diameter of 300 nm (FIGS. 14(a) and 14(b)). The observed particle diameter far exceeded the crystal grain size calculated from XRD revealing the particles to be polycrystalline in nature. Synthesized Co particles were observed to agglomerate heavily into secondary particles several microns in diameter (FIG. 14(c)). High resolution TEM imaging revealed the presence of interesting crystallographic features in the Co particle system. Lattice spacings of 1.28 nm were observed in select Co particles (FIG. 14(d)). The observed lattice spacings for the Co particles are significantly larger than lattice spacing for the Co (FCC or HCP) allotropes and are indicative of long range crystallographic ordering. The common observance of stacking faults in Co particles containing the FCC and HCP phases is a possible explanation for the large lattice spacings, however at this time further investigation is required to better understand this intriguing crystallographic ordering.¹⁴ Inspection of the Ni and Co particle's surface showed very little presence of an organic capping layer which is commonly witnessed when employing higher boiling point solvents. However, thermogravimetric analysis identified the presence of volatile organic adsorbates resulting in a 2.5% weight decrease when heating the particles to 200° C. A typical TGA curve is shown in FIG. 15.

Room temperature magnetization versus applied field (M(H)) curves show the Ni and Co particles to possess magnetic saturation (Mₛ) values of 51 emu/g and 148 emu/g, respectively (FIG. 16(a)). These Mₛ values compare well to the bulk magnetization of 58 emu/g for Ni and 163 emu/g for Co.¹⁶ Coercivity values of 180 Oe were recorded for the Co and Ni systems. For the Ni particles, this coercivity is increased over Ni bulk value; however this is common for magnetic nanomaterials as the crystal grain size approaches their critical magnetic domain size.¹⁷⁻¹⁹ For the Co particles, the presence of the hard ferromagnetic HCP—Co phase also causes an increase in coercivity over pure FCC—Co.²⁰ Zero field cooled (ZFC) curves for Ni and Co exhibited magnetic blocking events below room temperature, again consistent with crystal grain sizes approaching the critical magnetic domain size. Field cooled (FC) magnetization versus temperature plots for the Ni and Co particles show little variance in magnetic susceptibility from 50 K to 300 K, indicative of a strongly interacting ferromagnetic system²¹ (FIG. 16(b)). The ZFC curve for Co shows an inflection point at 160 K, which is common for Co nanomaterials. This phenomena can be attributed to overcoming the Neél temperature of CoO.¹⁷ This confirmation of a slight amount of CoO coupled with the small CoPt alloy formation and organic adsorbates all help explain the slight decrease in Mₛ values for the as synthesized Co particles when compared to bulk values.

The purpose of this report was to investigate the effectiveness of ethanol as a solvent for the assisted reduction and formation of Co and Ni nanocrystalline particles. To induce particle formation, K₂PtCl₄ was utilized as a nucleating agent to promote heterogeneous nucleation. XRD revealed the Ni and Co particles to be nanocrystalline in nature. TEM showed that while nanocrystalline in nature, the Ni and Co formed polycrystalline particles of 300 nm and 500 nm and greater in diameter, respectively. Magnetization values for the synthesized Ni and Co particles compare well with bulk magnetizations.¹⁶ This report demonstrates that low boiling point primary alcohols can acts as efficient solvents for the solvent assisted reduction of ferromagnetic Co and Ni nanocrystalline particles. With the use of low boiling point solvents, such as ethanol, a more cost efficient and environmental friendly process for the synthesis of ferromagnetic nanocrystalline particles can be realized.

### REFERENCES FOR EXAMPLE 9

(1) Willard, M. A.; Kurihara, L. K.; Carpenter, E. E.; Calvin, S.; Harris, V. G. International Materials Reviews 2004, 49, 125-170.
(2) Carroll, K. J.; Reveles, J. U.; Shultz, M. D.; Khanna, S. N.; Carpenter, E. E. Journal of Physical Chemistry C 2010, 115, 2656-2664.
(3) Chakroune, N.; Viau, G.; Ricolleau, C.; Fievet-Vincent, F.; Fievet, F. Journal of Materials Chemistry 2003, 13, 312-318.
(4) Sun, S. H.; Murray, C. B.; Weller, D.; Folks, L.; Moser, A. Science 2000, 287, 1989-1992.
(5) Ung, D.; Soumare, Y.; Chakroune, N.; Viau, G.; Vaulay, M. J.; Richard, V.; Fievet, F. Chemistry of Materials 2007, 19, 2084-2094.
(6) Ung, D.; Viau, G.; Ricolleau, C.; Wannont, F.; Gredin, P.; Fievet, F. F. Advanced Materials 2005, 17, 338-+.
(7) Viau, G.; Garcia, C.; Maurer, T.; Chaboussant, G.; Ott, F.; Soumare, Y.; Piquemal, J. Y. Physica Status Solidi a-Applications and Materials Science 2009, 206, 663-666.
(8) Fievet, F.; Lagier, J. P.; Blin, B.; Beaudoin, B.; Figlarz, M. Solid State Ionics 1989, 32-3, 198-205.
(9) Hinotsu, T.; Jeyadevan, B.; Chinnasamy, C. N.; Shinoda, K.; Tohji, K. Journal of Applied Physics 2004, 95, 7477-7479.
(10) Bayram, B.; Soykal, I. I.; von Deak, D.; Miller, J. T.; Ozkan, U. S. Journal of Catalysis 2011, 284, 77-89.
(11) Choi, H.; Veriansyah, B.; Kim, J.; Kim, J. D.; Kang, J. W. Journal of Supercritical Fluids, 52, 285-291.
(12) Kim, J.; Kim, D.; Veriansyah, B.; Kang, J. W.; Kim, J.-D. Materials Letters 2009, 63, 1880-1882.
(13) Victor Antonio de la Pena, O. S.; Iberio de, P. R. M.; Alberto, R.; Francesc, I. The Journal of Chemical Physics 2010, 133, 024701.
(14) Dureuil, V.; Ricolleau, C.; Gandais, M.; Grigis, C. European Physical Journal D 2001, 14, 83-88.
(15) Pearson, W. B. Handbook of Lattice Spacings and Structures of Metals; Pergamon Press: London, 1967; Vol. 2.
(16) CRC Handbook of Chemistry and Physics; 55 ed.; CRC Press: Cleveland, Ohio, 1974.
(17) Gangopadhyay, S.; Hadjipanayis, G. C.; Sorensen, C. M.; Klabunde, K. J. Magnetics, IEEE Transactions on 1992, 28, 3174-3176.
(18) Gong, W.; Li, H.; Zhao, Z. G.; Chen, J. C. Journal of Applied Physics 1991, 69, 5119-5121.
(19) Du, Y. W.; Xu, M. X.; Jian, W.; Shi, Y. B.; Lu, H. X.; Xue, R. H. Journal of Applied Physics 1991, 70, 5903-5905.
(20) Sato, H.; Kitakami, O.; Sakurai, T.; Shimada, Y.; Otani, Y.; Fukamichi, K. Journal of Applied Physics 1997, 81, 1858-1862.
(21) Dormann, J. L., Fiorani, D Magnetic Properties of Fine Particle Systems; North-Holland: Amsterdam, 1992.

### Continuous Flow Synthesis of Nanoparticles

In continuous flow methods, one or more solutions which contain some or all of the reactants required for a desired chemical synthetic reaction to occur are provided. The one or more solutions are then introduced into a continuous flow device and subjected to a change in conditions. The change causes or allows the desired chemical reaction to proceed. For example, if a single (only one) solution is used, the solution is introduced into the device and one or more parameters (e.g. temperature, pressure, etc.) are altered and/or manipulated upon entry or thereafter to cause the desired reaction to take place. If two or more solutions are used, each of the solutions may be different and may contain a subset of the reactants needed to conduct the desired reaction, and/or to make the reaction move forward in a desired manner, or at a desired rate, etc. Upon introduction into the continuous flow device, the solutions mix and reaction is initiated under controlled and/or optimized conditions. For example, the solvents, the concentrations of reactants in each solution, the pH, etc. of the at least two solutions can be precisely and separately controlled; the rate at which the two or more solutions mix (e.g. the rate of entry or the flow rate of each solution into a common mixing chamber) can also be controlled, as can the temperature, pressure, residence time in the reactor, and other conditions of flow and mixing.

If two or more solutions are employed, generally a first solution is provided in which one or more metal salts is/are dissolved in a solvent and a second solution containing the same or another suitable solvent without the metal salt(s) but with other active ingredients, is also provided. The two solutions are initially housed in separate chambers or containers (reservoirs, etc.), and are drawn into a common mixing chamber e.g. by pumping. Generally, a means for stirring or agitating the solutions as they mix is provided, e.g. within a mixing chamber itself, or at a junction of the two flow paths located just outside the mixing chamber (although in some embodiments, the flow rate may be sufficiently high to cause adequate mixing of the two solutions at the point where they encounter each other). The mixing chamber may include a heating source. The flow rate and/or the volume of the mixing chamber may be adjusted so as to provide a suitable residence time of the reactants within the mixing chamber so that the conversion of metal salts in solution to solid nanoparticles can proceed to completion, or to near completion e.g. at least about 50, 55, 60, 65, 70, 75, 80, 85, 90, or 95% or more completion. Typical mean residence times are in the range of from about 0 to about 20 minutes, e.g. about 1, 2, 3, 4, 5, 6, 7, 8, 9 or 10 minutes, or more (e.g. about 11, 12, 13, 14, 15, 16, 17, 18, 19 or 20 minutes).

In some embodiments (e.g. in a microfluidic reactor) the mixing chamber is a channel comprised of e.g. tubing, which may be straight or coiled but is typically sinuous, and which is generally placed on a support member. In other embodiments, the mixing chamber may be a single container of sufficient volume to receive the solution(s) and house the reaction mixture.

As the reaction progresses within the mixing chamber, metallic nanoparticles are formed and are released and/or collected by any of several methods. For example, the reaction may be stopped and the particles may be collected or separated from the reaction liquid e.g. by gravity, filtering, by centrifugation, magnetic separation, spray drying, sedimentation, sieving, etc. Alternatively, the particles may be continually removed from the reaction chamber or continually sequestered in a particular location within the reaction chamber as the reaction proceeds, e.g. by trapping them with a filter or screen, by magnetic attraction, etc, Separation of the nanoparticles from other solids in the reaction mixture may be accomplished e.g. using centrifugal techniques, using magnets, by sieving according to size, etc.

Unreacted or spent reaction liquid, which contains lower levels of metal salts than when introduced into the mixing chamber, may be removed from the system or may be recycled back in to the reactor.

Prior art microfluidic reactors are described, for example, in U.S. Pat. No. 7,615,169 (Strouse et al.) and US patent application 20100184928 (Kumacheva), the complete contents of both of which are hereby incorporated by reference. In some embodiments, these or similar continuous flow reactors may be used in the methods. However, in other embodiments, the continuous flow reactors that are employed have novel features that are designed to facilitate or optimize reactions, yields, etc. of alloy magnetic nanoparticles as described herein.

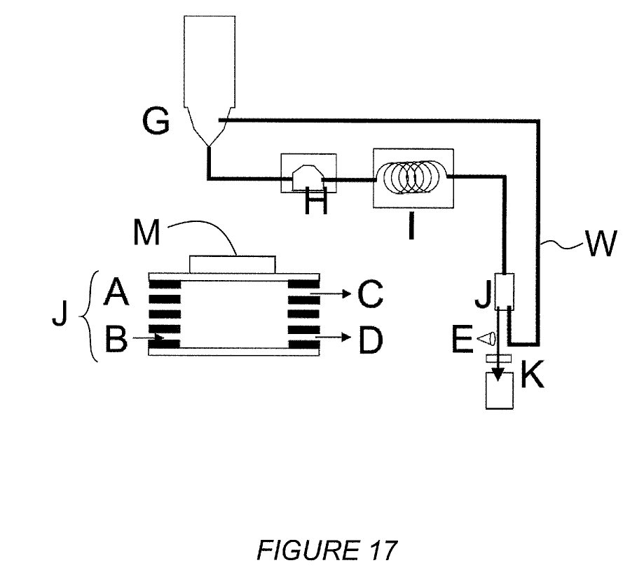

In some embodiments, the invention also provides a continuous flow system for carrying out the methods described herein. A schematic representation of an exemplary system is presented in FIG. 17. In FIG. 17, G is a reaction hopper (chamber, reservoir) which contains a metal salt dissolved in a solvent such as ethanol. H is a high pressure pump, I is a capillary microreactor which is heated to a desired temperature, J is a separator (shown in detail below the system), (e.g. a photodiode, magnetoresistance, hall probe, etc.) that detects (monitors) the particles and provides feedback to adjust the pump rate, temp etc., and K is a receptacle which receives the desired product. A-D represent the magnetic separator J which. A is a wash solvent, B is a reaction mixture, C is the desired product, and D is a waste stream. Magnet M is placed on the A-C side of the separator. The magnet pulls on and influences the flow path of magnetic particles, and the amount (strength) of the magnetic force determines which channel (e.g. top or bottom) metal particles enter when leaving the separator. For example, metal particles deflected toward the magnet are likely to enter the top channel and flow past detector E and into receptacle K. In contrast, unreacted liquid and waste components are not deflected, or are deflected to a lesser extent, and are likely to flow through the bottom channel upon egress from separator J, entering e.g. line (conduit, channel) W. In the embodiment shown in FIG. 17, the waste stream is recycled back into the reaction. This exemplary system can be readily adapted to a variety of continuous flow synthetic processes.

Generally, the system also includes a controller that controls (e.g. initiates, adjusts, and/or monitors, etc.) the conditions within the reactor, e.g. the temperature, pH, time of mixing, residence time, pressure, flow rates, mixing speed, volumes, etc. The controller is generally a computer that has been programmed to carry out instructions needed to implement the method steps, and/or to receive input from a human and from the various components of the system, and to provide output to the various components of the system and/or to a display device, as well as to monitor input on an ongoing basis. The computer is also generally programmed to conduct relevant processing, e.g. various calculations of data, etc. The instructions may reside on a non-transient medium such as a CD, DVD, flash drive, a hand held device, etc. and/or may be downloadable via the internet. Output from the controller may be displayed, e.g. on a computer monitor or other display screen, and/or may be printed out and provided as a hard copy.

### Adaptation of Batch Synthetic Processes to Continuos Flow

In some embodiments of the invention, a scalable wet chemical batch technique is adapted for use in continuous flow. The method used typically involves nucleation of one or more types of metal ions e.g. on a “scaffolding” or support provided by a reactant, followed by a growth phase of the metal on the scaffolding or support to form alloy nanoparticles. Depending on the solvent components that are utilized, the resulting nanoparticles may be, for example, carbides, nitrides, borides, phosphides, or sulfides or a mixture of these. Such particles demonstrate properties not found in the original bulk materials.

In one embodiment, the wet chemical technique is a polyol process in which a polyhydric alcohol (polyol) is base deprotonated to a glycolate which promotes reduction and nucleation of the metal salt, especially under high temperatures. The polyol functions as a solvent and reducing agent whereby a metal precursor is reduced to form metal nuclei attached to the glycolate. The nuclei grow on the glycolate through a traditional Ostwald ripening mechanism. The reaction is thus carried out under conditions which in effect allow ligand exchange to occur between the deprotonated alcohol and a metal salt of interest. At elevated temperatures, excess glycolate ions assist in the reduction of the metal, and also act as a capping agent. By controlling the reaction temperatures, pressures, and alkalinity, the reduction, nucleation, and growth dynamics of the reaction can be controlled to achieve a desired nanoparticle composition.

In some exemplary embodiments, pure Co₂C phase and pure Co₃C phase magnetic nanoparticles are formed in this manner (for details see Example 5).

In addition, it has been found that, with polyol processes, superior results are obtained with respect to nanoparticle yield and purity when the reaction is carried out by the incremental addition of small aliquots of the metal salt to the polyol at spaced apart time intervals, rather than adding the entire amount of metal salt to be reacted all at once to the reaction mixture. Without being bound by theory, it appears that the repeated (repetitive) addition of small amounts of the metal salt slows the growth of the material. Slowing growth may be key to forming the carbide phases with desired phase composition, size, and shape. The gradual addition of the metal salt allows sufficient time for reconstruction and/or diffusion of C atoms into the Co structure and the formation of the desired material, without impurities. Continuous flow methods are thus well-suited to such synthetic processes.

Polyol based synthesis of such nanoparticles typically involves the mixing of a polyol with at least one metal salt of interest. In some embodiments, a single type of metal salt is used. In other embodiments, two or more types of metal salts are used, resulting in production of nanoparticles with mixed metal compositions. Suitable polyols for use in the methods include but are not limited to, for example, various alcohols which have between 1 and 20 carbons, various di- or tri-alcohols; ethers with a terminal alcohol, and others.

Metals that may be employed include but are not limited to, for example, cobalt, iron, nickel, manganese, chromium, or their alloys. The metals are generally in the form of a salt formed with anions such as e.g. CH₃COO⁻, CO₃²⁻, Cl⁻, F⁻, HOC(COO⁻), (CH₂COO⁻)₂, C≡N⁻, NO₃⁻, NO₂⁻, PO₄³⁻, and SO₄²⁻, or organometallics like CO or C₆H₅. Metal alloys may also be used, examples of which include but are not limited to: CoNi, CoFe, NiFe, MnFe, CoNiFe, CoMnFe, etc

In yet other embodiments, one or more surfactants are included in one or both of the solvents. Exemplary solvents that may be used include but are not limited to: bis(2-ethylhexyl) sodium sulfosuccinate (AOT)-isooctane, nonylphenyl polyethoxylates such as Igepal Co-430™; polyvinyl alcohols such as polyvinylpyrrolidine (PVP), cetyltrimethylammonium bromide, and polyethylene glycols (PEGs)

Conditions for carrying out the reaction may vary depending on the metal(s) or alloys thereof that are used to form the nanoparticles. For cobalt-based nanoparticles, the [OH⁻] may be low (e.g. in the range of from about 0 to about 0.1M and usually from about 0 to about 0.05M to bias the reaction toward Co₃C, or may be high (e.g. in the range of from about 0.2 to about 1M and usually from about 0.3 to about 0.6) to bias the reaction toward Co₂C. For other metals, conditions are adjusted to achieve a desired level of polyol deprotonation, the desired level being that which permits the reaction to proceed at a suitable rate. For example Ni₃C can be synthesized at [OH⁻] in the range of 0M to 0.4M and usually from 0.14M to about 0.2M. Levels may be adjusted, e.g. by adjusting the pH of the reaction mixture by sodium or potassium hydroxide or sodium ethoxides, other suitable pH altering agents. For cobalt-based nanoparticles, the [Co⁺²] may be wide (e.g. in the range of from about 0.0001M to the solubility limit, and is usually from about 0.01 to about 0.1) to bias the reaction toward Co₃C, or may be low (e.g. in the range of from about 0.5 mM to about 2 mM, and usually from about 1 mM to about 1.5 mM) to bias the reaction toward Co₂C.

In addition, the rate of mixing the metal salt with the polyol is adjusted to provide a desirable reaction rate. Generally, the metal salt is added at a rate of from about 0.2 to about 0.4 mmoles per mL of glycol every 1 to 2 minutes.

The reaction may be carried out at a wide range of suitable temperatures, e.g. in the range of from about 180° C. to about 325° C., and usually from about 250° C. to about 325° C., depending on e.g. the desired rate of reaction, the reactants that are employed, and the products that are desired etc.

### Continuous Flow Synthesis of Nanoparticles Using Supercritical Fluids

In some embodiments, the continuous flow synthesis methods are carried out or performed using supercritical fluids (SCFs). The use of supercritical solvents in the continuous flow processes of the invention allows not only the efficient synthesis of nanoparticles, but also advantageously permits the rapid separation of the nanoparticles from the reaction mixture once the applied pressure is released.

Generally, the synthetic methods are carried out as described for continuous flow reactions above, except that the continuous flow system is pressurized during the reaction that forms the nanoparticles. Basically, instead of conducting the reaction in e.g. ethanol at low pressures in a manner similar to the polyol synthesis described above, the pressure is increased sufficiently to convert the ethanol to a SCF. Upon completion of the reaction, release of the pressure results in rapid evaporation of the SCF, leaving behind a dry powder that is or contains the alloy nanoparticles. In other words, the SCF flash vaporizes leaving a dry powder behind.

In some embodiments, the supercritical fluids that are used include: alcohols, examples of which include but are not limited to: ethanol, propanol, butanol, ethoxyethanol, etc.; various glycols such as those listed above; liquid carbon dioxide; and acetonitrile, etc. Basically, any liquid in which a metal salt of interest can be dissolved and which becomes a SCF under pressures that are attainable in a continuous flow reactor, may be used. Preferably, such reagents are advantageously inexpensive, and can also be easily recaptured upon removal from the reaction mix, allowing them to be reused. This further decreases the cost of manufacturing the nanoparticles and provides environmental advantages since waste disposal is minimized.

Metals which may be used include those listed above in the section entitled “Adaptation of Batch Synthetic Processes to Continuous Flow”.

### Exemplary Applications of the Technology

The magnetic nanomaterials of the invention may be used in many applications, including without limitation the fabrication of permanent magnets. In one embodiment, the invention thus provides permanent magnets formed from (i.e. which include or incorporate) the magnetic nanoparticles described herein, i.e. permanent magnets that do not contain rare earth metals (they are “non-rare earth” permanent magnets) and may be used in many different ways.

One exemplary application of nanoparticles according to exemplary embodiments of the invention is devices providing mechanical work and/or the conversion of electrical energy to mechanical energy. For example, permanent magnets comprising nanoparticles (e.g. Co₃C nanoparticles) may be incorporated into magnetic torque devices, electric/electromagnetic motors (e.g. in plug-in hybrid or other electric vehicles, or any other suitable application of electric motors), direct drive wind turbine power systems, various energy storage systems (e.g. flywheels), magnetic separation devices, magnetic holding devices, magnetic latches, magnetic bearing devices, meters, loudspeakers, relays, actuators (e.g. linear actuators, rotational actuators), eddy current brakes, and other products e.g. in toys, as fastening devices, as refrigerator magnets, etc.

In yet other exemplary embodiments, nanoparticles and permanent magnets may be included in applications and devices which convert mechanical energy to electrical energy. Examples include but are not limited to generators (e.g. magnetos, alternating-current generators that contain a permanent magnet), and alternators.

In an exemplary embodiment, a motor, generator, or combined motor/generator is provided having permanent magnets comprising a plurality of transition metal carbide nanoparticles. The nanoparticles are provided in accordance with the teachings herein. In particular, the nanoparticles used in the motor may an have an effective magnetocrystalline anisotropy constant (K_{eff}) in the boundary inclusive range of 6.5×10⁵ J/m³ to 9×10⁵ J/m³ and a critical temperature of at least 300 K. The motor or generator includes such components as a stator and a rotor. Features of these elements and other elements which may optionally be included in embodiments for motors and generators are disclosed in, e.g., U.S. Pat. No. 3,935,487 to Czerniak, US Patent Application Publication No. 2013/0249343 by Hunstable, and U.S. Pat. No. 381,968 to Nikola Tesla, and U.S. Pat. No. 8,310,126 to Hopkins et al., all of which are herein incorporated by reference.

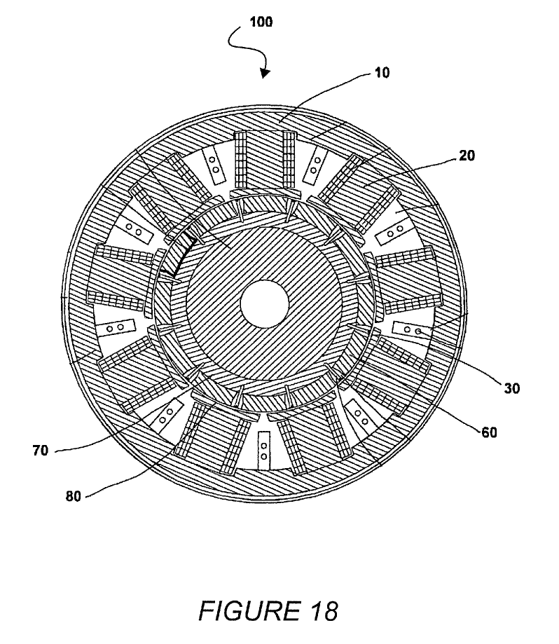

Referring now to FIG. 18, there is shown the stator and rotor assemblies of a motor/generator 100 in accordance with one example embodiment. There is shown a stator flux ring 10, a plurality of separate metal cores 20 wound as individual electromagnets and positioned immediately adjacent to and in contact with the stator flux ring 10, and a like plurality of shoes 30 to diffuse the magnetic flux field produced by each of the electromagnets. The metal stator flux ring 10, metal cores 20, and metal shoes 30 may be fabricated as a single component, or alternatively, may be made as separate components to facilitate their manufacture using molds and presses and to facilitate machine winding of the cores 20 to form electromagnets. The stator flux ring 10 provides a path for the magnetic flux field between the electromagnets. The shoes 30 are slanted in order to reduce cogging torque as the permanent magnets 60, mounted on the outer cylindrical surface of the rotor flux ring 70, move from one of the stator electromagnets to the next. The rotor assembly of motor/generator 100 is sized to provide an air gap 80 between the inner face of each of the electromagnet shoes 30 and the outer face of each of the permanent magnets 60. Permanent magnets 60 comprise nanoparticles according to the teachings herein. In particular, the nanoparticles may have an effective magnetocrystalline anisotropy constant (K_{eff}) in the boundary inclusive range of 6.5×10⁵ J/m³ to 9×10⁵ J/m³ and a critical temperature of at least 300 K.

Still further embodiments may employ nanoparticles and permanent magnets to shape or control electron or ion beams. Examples include but are not limited to cathode-ray tubes, traveling wave tubes, magnetrons, BWO's, Klystrons, ion pumps, and cyclotrons.

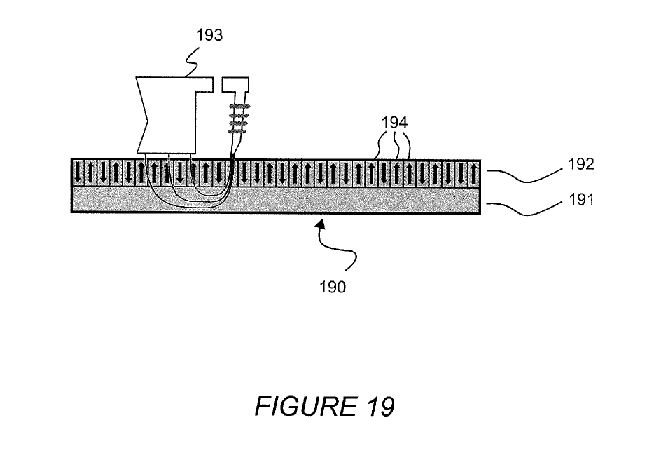

Nanoparticles according to exemplary embodiments of the invention are especially well suited for applications and devices where minimization of size and space requirements is desirable. In particular, nanoparticles according to those taught herein may be used in magnetic recording media (e.g. hard disks, hard disk drives (HDDs), floppy disks, magnetic tapes, etc.) In reference to the schematic of FIG. 19, a magnetic storage medium 190 such as a hard disk generally comprises at least a substrate 191 and a magnetic film 192. The magnetic storage medium 190 may be included in a magnetic storage system having additional elements such as a read/write head 193 for changing and/or reading the dipole moment of each nanoparticle 194. The bidirectionality of the dipole moment is used to store a bit of information. In binary, a first direction to the dipole moment represents a “0” and a second direction opposite the first direction represents a “1”. In an exemplary embodiment, the magnetic film 192 comprises a plurality of single phase magnetic alloy nanoparticles 194 having an effective magnetocrystalline anisotropy constant (K_{eff}) in the boundary inclusive range of 6.5×10⁵ J/m³ to 9×10⁵ J/m³ and a critical temperature of at least 300 K. The critical temperature may be at least 400 K, at least 500 K, or at least as high as 571 K. Provided the exceptionally high k_{eff} and critical temperature of the nanoparticles, the data density of the magnetic recording medium is higher than could be achieved with existing nanomagnets. Methods for the manufacture of magnetic storage media, additional elements usable therewith, and additional optional features therefore are disclosed in, e.g., U.S. Pat. No. 8,559,131 to Masuda et al.; U.S. Pat. No. 6,556,375 to Obara; US Patent Application Publication No. 2004/0161576 by Yoshimura; US Patent Application Publication No. 2004/0257711 by Ushiyama et al.; and U.S. Pat. No. 8,468,682 to Zhang, all of which are incorporated herein by reference and features of which may employed for embodiments in accordance with the present invention.

Nanoparticles according to exemplary embodiments of the invention may also be used in various biomedical applications. As one particular example, nanoparticles according to the invention may be used for a system or method for magnetic fluid hyperthermia. Generally, magnetic fluid hyperthermia is a method of medical treatment by which the temperature of a patient's body (the entire body or a localized area of a patient's body) is artificially elevated. Patients on which magnetic fluid hyperthermia may be used include both humans or animals. When sick or affected by some affliction (e.g. a bacterial or viral infection, cancer, etc.) the body's natural response is to induce a fever, elevating the body temperature. Healthy cells can withstand temperatures up to about 42 degrees Celsius (approx. 108 degrees Fahrenheit), while many sick cells (e.g. cancer cells), bacteria, and viruses find such elevated temperatures inhospitable for growth, reproduction, or even survival.

Magnetic fluid hyperthermia artificially elevates a temperature in a patient body to provide benefits similar to natural fevers. Therapeutic temperature ranges induced by magnetic fluid hypothermia include up to 42° C. or even up to 45°. Methods of medical magnetic hyperthermia are described in U.S. Pat. No. 7,842,281 to Haik et al. which is herein incorporated by reference. In one aspect of the invention, methods of hyperthermia treatment of a patient in need thereof are provided. In one embodiment, a method includes administering to a patient a composition comprising magnetic nanoparticles according to any of the nanoparticles taught herein (e.g. Co₃C nanoparticles); and then exposing the magnetic nanoparticles in the patient to an alternating magnetic field effective to generate hysteresis heat in the nanoparticles. In an exemplary embodiment, a therapeutic/pharmaceutical composition is provided which comprises a pharmaceutically acceptable carrier and a plurality of single phase magnetic alloy nanoparticles having an effective magnetocrystalline anisotropy constant in the boundary inclusive range of 6.5×10⁵ J/m³ to 9×10⁵ J/m³ and a critical temperature of at least 300 K. The composition may be administered to a tumor or other diseased tissue in the patient. The nanoparticles may be coated by a biocompatible polymeric material. In another embodiment, the magnetic nanoparticles are administered to a cancerous tissue site and the cancerous tissue site is further treated with one or more therapeutic drugs, one or more therapeutic radiation treatments, or a combination thereof. Magnetic nanoparticles with or without one or more therapeutic drugs and one or more therapeutic radiation treatments may be encapsulated such as in the manner disclosed in U.S. Patent Application Publication No. 2004/0065969 by Chatterjee, et al. which is herein incorporated by reference.

In other embodiments, the invention provides soft magnets. The soft magnets of the invention may be used in any suitable device or for any suitable application.

Where definitions in any patent or publication incorporated by reference into the present application conflict with definitions presented explicitly and outright in the immediate disclosure (that is to say, not by an incorporation by reference), definitions in the immediate disclosure are to be used in interpreting the claimed subject matter.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

What is claimed is:

1. A composition comprising one or more transition metal carbide nanoparticles having a particle size of 10 nm or less and a critical temperature of at least 300 K.

2. The composition of claim 1, wherein said critical temperature is in the boundary inclusive range of 300 K to 571 K.

3. The composition of claim 2, wherein said critical temperature is in the boundary inclusive range of 400 K to 571 K.

4. The composition of claim 3, wherein said critical temperature is in the boundary inclusive range of 500 K to 571 K.

5. The composition of claim 2, wherein said one or more transition metal carbide nanoparticles substantially consist of cobalt carbide (Co₃C).

6. The composition of claim 2, wherein said one or more transition metal carbide nanoparticles have an effective magnetocrystalline anisotropy constant in the boundary inclusive range of 6.5×10⁵ J/m³ to 9×10⁵ J/m³.

7. The composition of claim 2, wherein said one or more transition metal carbide nanoparticles are single phase.

8. The composition of claim 2, wherein said one or more transition metal carbide nanoparticles have a particle size at least as small as 8 nm.

9. A device for converting between mechanical energy and electrical energy, comprising nanoparticles of a single phase magnetic alloy, said nanoparticles having an effective magnetocrystalline anisotropy constant in the boundary inclusive range of 6.5×10⁵ J/m³ to 9×10⁵ J/m³ and a critical temperature of at least 300 K.

10. The device of claim 9, wherein said device is an electromagnetic motor or generator and said nanoparticles form one or more permanent magnets within said electromagnetic motor or generator.

11. The device of claim 9, wherein said critical temperature is in the boundary inclusive range of 300 K to 571 K.

12. The device of claim 9, wherein said one or more transition metal carbide nanoparticles substantially consist of cobalt carbide (Co₃C).

13. The device of claim 9, wherein said one or more transition metal carbide nanoparticles have an effective magnetocrystalline anisotropy constant in the boundary inclusive range of 6.5×10⁵ J/m³ to 9×10⁵ J/m³.

14. The device of claim 9, wherein said one or more transition metal carbide nanoparticles have a particle size of 10 nm or less.

15. A magnetic storage medium, comprising:
a substrate; and
a magnetic film applied to said substrate, said magnetic film comprising a plurality of single phase magnetic alloy nanoparticles having an effective magnetocrystalline anisotropy constant in the boundary inclusive range of 6.5×10⁵ J/m³ to 9×10⁵ J/m³ and a critical temperature of at least 300 K.

16. The magnetic storage medium of claim 15, wherein said critical temperature is in the boundary inclusive range of 300 K to 571 K.

17. The magnetic storage medium of claim 15, wherein said plurality of transition metal carbide nanoparticles substantially consist of cobalt carbide (Co₃C).

18. The magnetic storage medium of claim 15, wherein said plurality of transition metal carbide nanoparticles have an effective magnetocrystalline anisotropy constant in the boundary inclusive range of 6.5×10⁵ J/m³ to 9×10⁵ J/m³.

19. The magnetic storage medium of claim 15, wherein said plurality of transition metal carbide nanoparticles have a particle size of 10 nm or less.

20. The magnetic storage medium of claim 15, wherein said plurality of transition metal carbide nanoparticles have a particle size at least as small as 8 nm.

21. A pharmaceutical composition, comprising:
a plurality of single phase magnetic alloy nanoparticles having an effective magnetocrystalline anisotropy constant in the boundary inclusive range of 6.5×10⁵ J/m³ to 9×10⁵ J/m³ and a critical temperature of at least 300 K; and
a pharmaceutically acceptable carrier.
